(19) United States
(12) Patent Application Publication
(10) Pub. No.: US 2010/0130434 A1
(43) Pub. Date: May 27, 2010

(54) Hexose Compounds to Treat Cancer

(75) Inventors: Waldemar Priebe, Houston, TX (US); Izabela Fokt, Houston, TX (US); Charles Conrad, Spring, TX (US); Sigmund Hsu, Houston, TX (US); Slawomir Szymanski, Spring, TX (US); Theodore Lampidis, Miami, FL (US)

Correspondence Address: Nielsen IP Law LLC, 1177 West Loop South, Suite 1600, Houston, TX, 77027, US

(73) Assignee: BOARD OF REGENTS, THE UNIVERSITY OF TEXAS SYSTEM, Austin, TX (US)

(21) Appl. No.: 12/280,322

(22) PCT Filed: Feb. 26, 2007
(86) PCT No.: PCT/US2007/062789

§ 371 Date: Jan. 20, 2010

Related U.S. Application Data
(60) Provisional application No. 60/776,793, filed on Feb. 24, 2006.
(60) Provisional application No. 60/795,621, filed on Apr. 27, 2006.
(60) Provisional application No. 60/796,173, filed on Apr. 28, 2006.

(51) Int. Cl. A61K 31/70

(52) U.S. Cl. 514/23

(57) ABSTRACT

Methods of treating glioblastoma and pancreatic cancer are provided by the administration of a therapeutically effective amount of a hexose compound to a subject in need thereof The subject invention includes methods of treating brain and pancreatic cancer comprising the administration of a therapeutically effective amount of a mannose compound to a subject in need thereof The subject invention further includes methods of treating the proliferation of tumors comprising the administration of a therapeutically effective amount of 2-FM to a subject in need thereof.

### CROSS-REFERENCE TO RELATED APPLICATIONS

This application claims priority to U.S. provisional application 60/795,621 filed Apr. 27, 2006[;] and to U.S. provisional application 60/796,173 filed Apr. 28, 2006. U.S. provisional applications, 60/795,621 and 60/796,173 are incorporated by reference herein in their entirety.

### STATEMENT REGARDING FEDERALLY SPONSORED RESEARCH OR DEVELOPMENT

This invention was made with government support under CA101936 awarded by National Institute of Health. The government has certain rights in the invention.

### THE NAMES OF PARTIES TO A JOINT RESEARCH AGREEMENT.

None.

### INCORPORATION-BY-REFERENCE OF MATERIAL SUBMITTED ON A COMPACT DISC

None.

### FIELD OF INVENTION

The present invention is directed to hexose compounds useful in the treatment of cancer and methods of treating of cancer-mediated diseases in a subject in need thereof by administering such compound.

### BACKGROUND OF THE INVENTION

Treatments of cancer are often associated with the challenges of the development of tumor resistance. Apoptosis, a type of programmed cell death, involves a series of biochemical events that lead to cell morphology and death. The apoptotic process is executed in such a way as to safely dispose of cell fragments. By elucidating intracellular signal transduction pathways through cancer therapy, however, it is possible for the structures and processes crucial for induction of cell death to be affected. Indeed, defective apoptosis processes have been implicated in numerous diseases. Excess apoptosis causes cell-lose disease like ischemic damage. On the other hand, insufficient amounts of apoptosis results in uncontrolled cell proliferation such as cancer.

Changes occur with the progression of malignant gliomas may be related to the activation of the PI-3K/AKT pathway (typically by PTEN loss or through growth factor activity such as EGFR). This survival pathway activates a number of adaptive changes that include among other things, a stimulus for angiogenesis, inhibitors to apoptosis, and metabolic shifts that promote activation of glycolysis, preferentially. Similarly, new targets of treatment for pancreatic cancer include targets of signal transduction pathways and molecules involved in angiogenesis, specifically, the ras oncogene signally pathway and inhibitors of the matrix metalloprotease (MMP).

Many cancers such as malignant gliomas and pancreatic cancer are intrinsically resistant to conventional therapies and represent significant therapeutic challenges. Malignant gliomas have an annual incidence of 6.4 cases per 100,000 (Central Brain Tumor Registry of the United States, 2002-2003) and are the most common subtype of primary brain tumors and the deadliest human cancers. In its most aggressive manifestation, glioblastoma multiforme (GBM), the median survival duration for patients ranges from 9 to 12 months, despite maximum treatment efforts. In fact, approximately one-third of patients with GBM their tumors will continue to grow despite treatment with radiation and chemotherapy. Similarly, depending on the extent of the tumour at the time of diagnosis, the prognosis for pancreatic cancer is generally regarded as poor, with few victims still alive 5 years after diagnosis, and complete remission rare.

Further, in addition to the development of tumor resistance to treatments, another problem in treating malignant tumors is the toxicity of the treatment to normal tissues unaffected by disease. Often chemotherapy is targeted at killing rapidly-dividing cells regardless of whether those cells are normal or malignant. However, widespread cell death and the associated side effects of cancer treatments may not be necessary for tumor suppression if the growth control pathways of tumors can be disabled. For example, one approach is the use of therapy sensitization, i.e. using low dose of a standard treatment in combination with a drug that specifically targets crucial processes in the tumor cell, increasing the effects of the other drug.

Furthermore, combination therapies include vaccine based approaches in combination with the cytoreductive and immune-modulating elements of chemotherapy with the tumor cell cytotoxic specificity of immunotherapy. Combination therapies, however, are typically more difficult for both the patient and physician than therapies requiring only a single agent. Furthermore, certain tumors have an intrinsic resistance against radiotherapy and many chemotherapy modalities may be due to the differential growth patterns and different types of growth patterns can represent various degrees of hypoxic regions within individual tumors. For example, gliomas can grow in predominately infiltrative fashion with little to no contrast enhancement seen on MRI scans versus more rapidly growing contrast enhancing mass lesions. Similarly, the early stages of pancreatic cancer can go undetected. Also, relative hypoxic areas can be seen both in the center of the rapidly growing tumor mass, which often has regions of necrosis associated with this, as well as some relatively hypoxic regions within the infiltrative component of the tumor as well. Accordingly, some of these relatively hypoxic regions may have cells, which are cycling at a slower rate and may therefore be resistant to chemotherapy agents.

Recently, certain proposed cancer therapies target the use of glycolytic inhibitors. This type of inhibitor is designed to benefit from the selectivity resulting when a cell switches from aerobic to anaerobic metabolism. Because of the growth of the tumor, cancer cells become removed from the blood (oxygen supply). Under hypoxia, the tumor cells up-regulate expression of both glucose transporters and glycolytic enzymes, in turn, favoring an increased uptake of the glucose analogs as compared to normal cells in an aerobic environment. Blocking glycolysis in a cell in the blood will not kill the cell because the cell survives by using oxygen to burn fat and protein in their mitochondria to produce energy (via energy-storing molecules such as ATP). By contrast, when glycolysis is blocked in cells in a hypoxic environment, the cell dies, because without oxygen, the cell is unable to produce energy via mitochondria) oxidation of fat and protein. Hence, while glycolytic inhibitors have shown promise to treat certain cancers, not all cancer cells exist in a hypoxic environment. Indeed, classic observations by Otto Warburg have demonstrated a preference of many tumors to preferentially utilize glycolysis for cellular energy production, even in the presence of adequate amounts of oxygen (termed oxidative glycolysis or the “Warburg effect”). This tumor adaptive response appears to hold true for malignant gliomas as well.

A need exists, therefore, for the treatment of cancers that show a resistance to chemotherapy, exhibit differential growth patterns or growth patterns that have various degrees of hypoxic regions within the tumor and/or have survival pathways which are a stimulus for angiogenesis or inhibit apoptosis.

### BRIEF SUMMARY OF THE INVENTION

Hexose compounds and pharmaceutical compositions thereof that prevent, inhibit and modulate cancer have been found, together with using the compounds for treatment of cancer, particularly, glioblastoma and pancreatic cancer. The present invention discloses the use of hexose compounds useful in treating cancer and cancer-mediated disorders and conditions. Methods of treatment of glioblastoma and pancreatic cancer comprise the administration of a therapeutically effective amount of a hexose compound to a subject in need thereof. Of particular interest is the method of treating the proliferation of tumors comprising the administration of a therapeutically effective amount of 2-FM to a subject in need thereof. The present invention includes methods of treating cancer by administering a mannose compound to a subject in need thereof.

### BRIEF DESCRIPTION OF THE SEVERAL VIEWS OF THE DRAWINGS

FIG. 1A depicts the results of a tumor growth inhibitory assay in SKBR3 cells with 2-DG, 2-FDG, 2-FDM and oxamate over a period of 24 hrs. Each value is the average±SD of triplicate samples.

FIG. 1B depicts the results of a cytotoxic assay in SKBR3 cells with 2-DG, 2-FDG, 2-FDM or oxamate and 24 hrs. Each value is the average±SD of triplicate samples.

FIG. 2A depicts the results of SKBR3 cell growth for 24 hr in the absence or presence of either 2 mM of 2-DG or 2-FDG and lactate concentration in the medium.

FIG. 2B depicts the results of SKBR3 cell growth for 6 hours in the presence of either 2-DG or 2-FDG at the same concentrations used in FIG. 2A followed by quantification of ATP in whole cell lysates.

FIG. 3A depicts the results of growth inhibitory assays in SKBR3 cells following treatment with 2-DG in the presence of various sugars. Each value is the average±SD of triplicate samples.

FIG. 3B depicts the results of cytotoxic assays in SKBR3 cells following treatment with 2-DG in the presence of various sugars. Each value is the average ±SD of triplicate samples.

FIG. 3C depicts the results of growth inhibitory assays in three different models of ‘hypoxia’ following treatment with 2-DG in the presence or absence of 2 mM mannose.

FIG. 3C depicts the results of cytotoxic assays in three different models of ‘hypoxia’ following treatment with 2-DG in the presence or absence of 2 mM mannose.

FIG. 4A depicts the results of SKBR3 cells treated for 48 hr with various drugs as indicated for each lane and total cell extracts were obtained and blotted with HRP-conjugated ConA. Equal amounts of protein were loaded in each lane and verified by B-actin. The glycoproteins (demarked by arrows) show that 8 mM of 2-DG and 2-FDM but not 2-FDG decrease their ConA binding and that this reduction can be reversed by mannose.

FIG. 4B depicts the results of the cells of FIG. 4A which were blotted for erbB2, a highly expressed glycoprotein. A change in the molecular weight of this protein is caused by similar doses of 2-DG and 2-FDM.

FIG. 5A shows the results of SKBR3 cells treated with 8 mM of either 2-DG, 2-FDG or 2-FDM for 24 hrs and whole cell lysates were blotted for two molecular chaperones, Grp78 and Grp94. 1 micro g/ml of tunicamycin (TUN) was used as a positive control. Protein loading was verified by β-actin.

FIG. 5B shows western blots of the proteins assayed when cells in models of “hypoxia” A, B & C were treated with similar doses of sugar analogs.

FIG. 6 depicts the results of SKBR3 cells treated with 8 mM of either 2-DG, 2-FDG or 2-FDM for 24 hrs and whole cell lysates were probed for CHOP/GADD154. Induction of CHOP/GADD154 induced by both 2-DG and 2-FDM was reversed by addition of exogenous mannose, whereas glucose showed no effect on the amount of this protein. Tunicamycin was used as a positive control. Protein loading was verified by β-actin.

FIG. 7 shows glycolysis and N-linked glycosylation pathways illustrate that 2-DG, 2-FDM and 2-FDG can inhibit phosphoglucoisomerase resulting in blockage of glycolysis and ensuing cell death in hypoxic tumor cells. However, in certain tumor cell types under aerobic conditions, 2-DG and 2FDM may interfere with lipid-linked assembly of oligosaccharides leading to induction of unfolded protein response and toxicity, because their structures resemble mannose as well as glucose. (triangle=glucose, hexagon=mannose and square=N-acetyl-glucosamine)

FIG. 8A shows MTT assays demonstrating the sensitivities of selected glioma cell lines and certain hexose compounds of the subject invention.

FIG. 8B shows MTT assays demonstrating the sensitivities of selected glioma cell lines and certain hexose compounds of the subject invention.

FIG. 8C shows MTT assays demonstrating the sensitivities of selected glioma cell lines and certain hexose compounds of the subject invention.

FIG. 9A depicts glioma cell growth upon treatment with various hexose compounds.

FIG. 9B depicts suppression of D54 cell growth upon treatment with 2-DG.

FIG. 9C depicts suppression of D54 cell growth upon treatment with 2-FG.

FIG. 10 demonstrates the difference in the effect of hypoxia on cells treated with 2-DG.

FIG. 11 shows the lactate production of a human glioblastoma cell line under hypoxic and normoxic conditions.

FIG. 12 shows results of glioma cell line growth under hypoxic and normoxic conditions.

FIG. 13 demonstrates the uptake of 2-FG in glioma cells.

FIG. 14 shows the results of treatment of gliomas in mice with 2-DG.

FIG. 15 shows 2-FM activity against Colo357-FG pancreatic cancer cells.

FIG. 16 shows 2-halo-D-mannose activity against U251 glioma cells.

FIG. 17 shows the suppression of U87 cell grown by 2-FM.

FIG. 18 provides a chart depicting the percent induction of autophagy in U87 glioma cells after treatment with 2-fluoro-mannose.

### DETAILED DESCRIPTION OF THE INVENTION

Therapeutic options for malignant gliomas remain quite limited. This is due in part to the intrinsic resistance of the cells to many chemotherapy options that are available. It may also be due in part to the differential growth patterns which malignant gliomas exhibit. Namely, gliomas can grow in predominately in infiltrative fashion with little to no contrast enhancement seen on MRI scans versus more rapidly growing contrast enhancing mass lesions. Many studies have indicated that these different types of growth patterns also represent various degrees of hypoxic regions within individual tumors. Relative hypoxic areas can be seen both in the center of the rapidly growing tumor mass, which often has regions of necrosis associated with this, as well as some relatively hypoxic regions within the infiltrative component of the tumor as well. Accordingly, some of these relatively hypoxic regions may have cells, which are cycling at a slower rate and may therefore be more resistant to many chemotherapy agents. Additionally, the observations by Warburg who described a preference of many tumors to undergo glycolysis even in the presence of adequate amounts of oxygen (termed oxidative glycolysis or the “Warburg effect”) appears to hold true for malignant gliomas as well. We postulated that because of these features, gliomas and other highly glycolytically sustained tumors such as pancreatic cancer may be sensitive to inhibitors of glycolysis and may have a significant impact on the tumor growth.

Hence, additional features unique to the brain generally, and gliomas specifically, is the increased expression of glucose transporters, which produces avid uptake of sugar into the CNS. We postulated that because of these features, gliomas represent a unique disease state that should be particularly sensitive to inhibitors of glycolysis. To test this hypothesis we used known inhibitors of glycolysis against a number of glioma cell line panels in vitro under both hypoxic and normoxic conditions. The effect of the agents were also examined in animals bearing orthotopic glioma xenografts using a number of different dosing schemes.

A shift in metabolism by high-grade gliomas to preferentially utilize glycolysis as the primary source for energy production even in the presence of oxygen “The Warburg Effect”, which is in part driven by HIF-1a and activation of the PI-3 kinase pathway. An effective inhibitor of glycolysis, 2-deoxyglucose blocks the conversion of 2-deoxyglucose-6-phosphate by the enolase reaction and produces an accumulation of this species in the cell due to the charged phosphate group.

Known metabolic shifts occur in high-grade neoplasms, including gliomas that preferentially use glycolysis for the energy requirements of the cell. These shifts are driven by survival pathways including HIF-1a and PI-3 kinase activation that induce production of critical enzymes required for glycolysis as well as up-regulate glucose transporters. This glycolytic phenotype is a dominant characteristic, which prevails even under normoxic conditions. This phenotype has been recognized and previously described as “The Warburg Effect”. Due to this phenotypic shift, these tumors should be more sensitive to inhibitors of glycolysis than normal cells. A group of sugar-based glycolitic inhibitors and other mannose compounds can serve as therapeutic agents. A prototypic sugar-based inhibitor 2-deoxyglucose has been shown to have tolerable and potent anti-glioma effects in this study. Hexose compounds either alone or in combination with cytotoxic chemotherapy are effective in treating cancer, particularly, gliomas and pancreatic cancer. Additionally, since this glycolytic phenotype is initially driven by hypoxic conditions within the tumor environment, this type of therapy should be considered with anti-angiogenic therapy. In fact, tumors that are capable of “escaping” anti-angiogenic therapy may be preferentially more sensitive to inhibitors of glycolysis and/or hexose compounds in general.

We have shown that sugar-based hexose compounds are efficacious in the treatment of high-grade glioma tumors and pancreatic cancer. Additionally, other inhibitor-type of compounds are being designed to have favorable uptake into the CNS and maintains the favorable oral bioavailability that 2-DG currently enjoys. Ongoing studies with hexose compounds both in combination with cytotoxic agents and anti-angiogenic agents, optimistically will provide intelligent leads for future clinical combinatorial trials.

A hexose compound means and includes any monosaccharide containing six carbon atoms. One class of hexoses is the aldohexose family, which includes glucose, galactose, and mannose, for example. The aldohexoses may also comprise various deoxysugars such as 2-deoxyglucose, fucose, cymarose, and rhamnose. Another class of hexoses is the ketohexose family exemplified by fructose and sorbose. Although hexoses of the present invention are normally of the naturally occurring D-configuration, the hexoses can also be L-enantiomers. Hexoses of the present invention may include alpha anomers, beta anomers, and mixtures thereof. Any of the hexoses of the present invention can be optionally substituted. Such substitutions involve replacement of a hydroxyl group with a halogen such as fluorine, chlorine, or bromine. In the present invention substitution is typically at the C-2 carbon of the hexose and may occupy either the axial or equatorial position of a hexose in its 6-membered ring chair conformation. Substitution at C-2 that is axial designates the sugar as a mannose derivative or a sugar of manno configuration. Substitution at C-2 that is equatorial designates the sugar as a glucose derivative or a sugar of gluco configuration.

Hexose compounds useful in the practice of the subject invention include compounds disclosed in U.S. Pat. No. 6,670,330 and U.S. Patent Applications 20030181393, 20050043250 and 20060025351, herein incorporated by reference. In certain embodiments of the present invention preferred compounds are sugar-based inhibitors of tumor proliferation such as 2-deoxy-glucose (2-DG), 2-deoxy-mannose (2-DM), 2-fluoro-glucose (2-FG) and 2-fluoro-mannose (2-FM) and the like.

“Tumor of the central nervous system” means any abnormal growth of tissue within the brain, spinal cord or other central-nervous-system tissue, either benign or malignant. It particularly includes gliomas such as pilocytic astrocytoma, low-grade astrocytoma, anaplastic astrocytoma and glioblastoma multiforme (GBM or glioblastoma). “Tumor of the central nervous system” also includes other types of benign or malignant gliomas such as brain stem glioma, ependymoma, ganglioneuroma, juvenile pilocytic glioma, mixed glioma, oligodendroglioma and optic nerve glioma. “Tumor of the central nervous system” also includes non-gliomas such as chordoma, craniopharyngioma, medulloblastoma, meningioma, pineal tumors, pituitary adenoma, primitive neuroectodermal tumors, schwannoma, vascular tumors and neurofibromas. Finally, Tumor of the central nervous system also includes metastatic tumors where malignant cells have spread to the central nervous system from other parts of the body.

According to the present invention “treating,” “treatment” or “alleviation” refers to both therapeutic treatment and prophylactic or preventative measures, wherein the object is to prevent or slow the growth of a tumor of the central nervous system, to reduce the size of tumor or to eliminate it entirely. Those in need of treatment include subjects having an identified tumor of the central nervous system, subjects suspected of having a tumor of the central nervous system and subjects identified as being at risk for the development of a tumor of the central nervous system. A subject is successfully “treated” for a tumor of the central nervous system if, after receiving a therapeutic amount of a hexose compound according to the methods of the present invention, one or more of the following conditions is observed: reduction in the size of the tumor or absence of the tumor; inhibition or cessation of growth of the tumor; inhibition or cessation of tumor metastasis; and/or relief to some extent of one or more of the symptoms associated with the tumor such as reduced morbidity and mortality or improved quality of life.

To the extent the hexose compound prevents growth and/or kill existing brain tumor cells, they may be considered cytostatic and/or cytotoxic.

The terms “coadministering” or “coadministration” are intended to encompass simultaneous or sequential administration of therapies. For example, co-administration may include administering both a glycolytic inhibitor and a chemotherapeutic agent in a single composition. It may also include simultaneous administration of a plurality of such compositions. Alternatively, coadministration may include administration of a plurality of such compositions at different times during the same period.

A hexose compound according to the present invention includes but is not limited to a glycolytic inhibitor which is a compound capable of inhibiting oxidative glycolysis in a glioma or other brain tumor and may include hexose compounds such as 2-deoxyglucose, 2-fluoro-glucose, 2-fluoro-mannose and the like.

The anti-proliferative treatment defined herein before may be applied as a sole therapy or may involve, in addition to at least one compound of the invention, one or more other substances and/or treatments. Such treatment may be achieved by way of the simultaneous, sequential or separate administration of the individual components of the treatment. The compounds of this invention may also be useful in combination with known anti-cancer and cytotoxic agents and treatments such as radiation therapy. If formulated as a fixed dose, such combination products employ the compounds of this invention within the dosage range described herein and the other pharmaceutically active agent within its approved dosage range. Glycolytic inhibitors may be used sequentially as part of a chemotherapeutic regimen also involving other anticancer or cytotoxic agents and/or in conjunction with non-chemotherapeutic treatments such as surgery or radiation therapy.

Chemotherapeutic agents includes, but is not limited to, three main categories of therapeutic agent: (i) antiangiogenic agents such as, linomide, inhibitors of integrin-alpha-beta 3 function, angiostatin, razoxane); (ii) cytostatic agents such as antiestrogens (for example, tamoxifen, toremifene, raloxifene, droloxifene, iodoxifene), progestogens (for example megestrol acetate), aromatase inhibitors (for example anastrozole, letrozole, borazole, exemestane), antihormones, antiprogestogens, antiandrogens (for example flutamide, nilutamide, bicalutamide, cyproterone acetate), LHRH agonists and antagonists (for example, gosereline acetate, leuprolide), inhibitors of testosterone 5-alpha-dihydroreductase (for example, fmasteride), farnesyltransferase inhibitors, anti-invasion agents (for example, metalloproteinase inhibitors like marimastat and inhibitors of urokinase plasminogen activator receptor function) and inhibitors of growth factor function, (such growth factors include for example, EGF, FGF, platelet derived growth factor and hepatocyte growth factor such inhibitors include growth factor antibodies, growth factor receptor antibodies such as Avastin. (bevacizumab) and Erbitux. (cetuximab); tyrosine kinase inhibitors and serine/threonine kinase inhibitors); and (iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); Intercalating antitumor antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide nitrosoureas, thiotepa; antimitotic agents (for example vinca alkaloids like vincristine and taxoids like Taxol (paclitaxel), Taxotere (docetaxel) and newer microbtubule agents such as epothilone analogs, discodermolide analogs, and eleutherobin analogs); topoisomerase inhibitors (for example epipodophyllotoxins like etoposide and teniposide, amsacrine, topotecan); cell cycle inhibitors; biological response modifiers and proteasome inhibitors such as Velcade (bortezomib).

One of ordinary still in the art will readily recognize that the methods of treatment disclosed in the present invention can be accomplished through multiple routes of administration and with various quantities/concentrations of hexose compounds. The preferred route of administration can vary depending on the hexose compounds being used and such routes include, but are not limited to, oral, buccal, intramuscular (i.m.), intravenous (i.v.), intraparenteral (i.p.), topical, or any other FDA recognized route of administration. The administered or therapeutic concentrations will vary depending upon the subject being treated and the hexose compounds being administered. In certain embodiments, the concentration of hexose compounds ranges from 1 mg to 50 gm per kilogram body weight.

Initially a series of 2-fluoro, 2-bromo, and 2-chloro-substituted glucose analogs was prepared and analyzed as possible competitive substrates to glucose in the glycolysis pathway, and that such analogs might operate as glycolytic inhibitors in a manner similar to 2-deoxy-glucose (2-DG). We have discovered that 2-fluoro-D-mannose is an effective antitumor agent because its properties might be derived from the fact that 2-fluoro-D-mannose (herein also referred to as “2-FM”) is either similar to 2-deoxy-D-glucose (same as 2-deoxy-D-mannose) considering the similarity in size of fluorine atom and hydrogen or it could be similar to D-mannose by resembling hydroxyl group of mannose better than hydrogen in terms of inductive effects and the possibility of hydrogen bonding formation. In a later situation 2-fluoro-D-mannose could affect biological functions, metabolism and biological processes related to D-mannose. Also, the combination of effects that could effect both D-glucose and D-mannose related cellular processes.

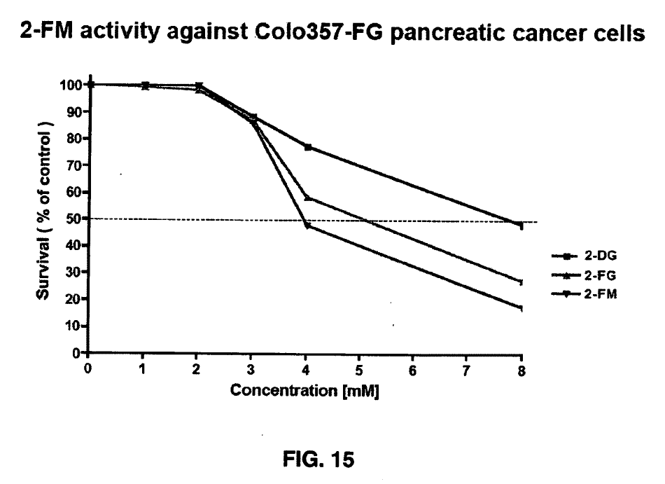

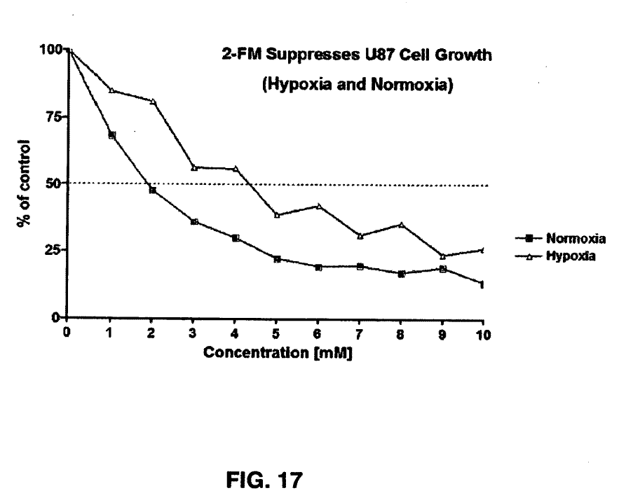

In fact, the data provided in FIGS. 15 through 18 shows that 2-fluoro-D-mannose is more potent than 2-DG and also has better or similar activity than that of 2-fluoro-D-glucose in pancreatic Colo357-FG cells. In addition, 2-fluoro-D-mannose (2-FM) was compared with other 2-deoxy-D-mannose analogs namely with 2-chloro-D-mannose (2-CM) and 2-bromo-D-mannose (2-BM). Surprisingly and not predicted, 2-fluoro-mannose is more potent than the others in this series. Specifically, the data shows that 2-fluoro-D-mannose (2-FM) is clearly superior to both bromo (2-BM) and chloro (2-CM) analogs in inhibiting growth of U251 glioblastoma brain tumor cells. 2-FM also displayed surprisingly better activity under normoxia than under hypoxia against U87 glioblastoma cells (FIG. 17). Additionally, at least one mode of action of 2-FM that was impossible to predict that is 2-FM displaying ability to potently induce autophagy in tumor brain cells and therefore provides at least one explanation of the mechanism of its action against tumor cell lines.

As shown immediately below, 2-deoxy glucose (2-DG) has two hydrogens at the C-2 position of the sugar. In the 6-membered ring chair conformation of the sugar, these two hydrogens occupy axial and equitorial positions.

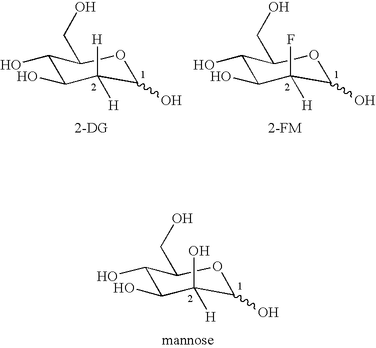

In essence, 2-fluoromannose (2-FM) replaces the axial hydrogen in 2-deoxyglucose (which is the same thing as 2 deoxymannose) with fluorine. Fluorine is generally considered isosteric with hydrogen. Thus, in some aspects, the chemistry of 2-FM might be similar to 2-DG. Indeed, 2-FM might exhibit glycolytic inhibitory activity based on this isoteric argument. However, fluorine is substantially more electronegative than hydrogen and is capable of engaging in hydrogen bonding motifs as a result. In this respect, 2-FM might behave more closely to mannose and, thus, 2-FM might disrupt the N-linked glycolipid/protein pathways in the synthesis of high mannose oligosaccharides.

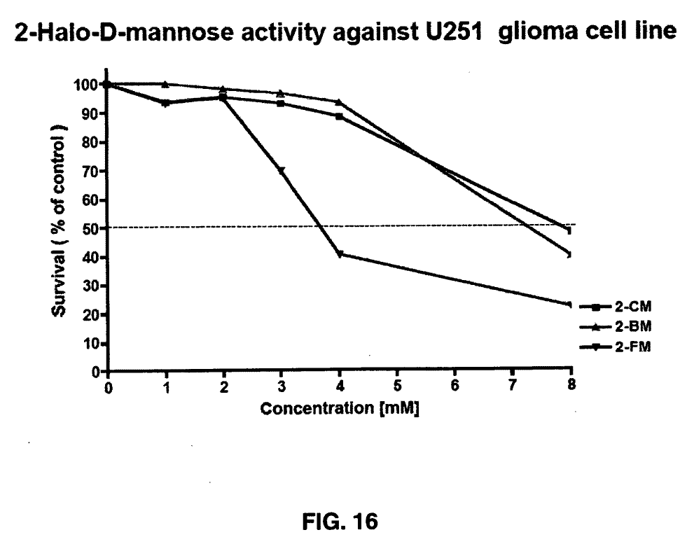

In short, 2-FM displays surprisingly good proliferating effects against tumor cells and appears more potent than 2-deoxy-D-glucose (also referred to herein as “2-DG”) and 2-deoxy-2-flouro-D-glucose (also referred to herein as “2-FG”). As discussed below, compound 2-FM was specifically tested in 231-GFP breast cancer, U251 glioblastoma multiforme brain tumor (FIG. 16) and Co1o357-FG pancreatic human cancer cell lines (FIG. 15). In U251 and Colo357-FG cells, 2-FM was directly compared with 2-DG, 2-FG, 2-deoxy-2chloro-D-mannose (herein referred to sometimes as “2-CM”), 2-deoxy-2-bromo-mannose (also referred to herein as “2-BM”), 2-deoxy-chloro-D-glucose (also referred to herein as “2-CG”) and 2-deoxy-2-brom-D-glucose (also referred to herein as “2-BG”). In both glioblastoma (FIGS. 16, 17 and 18) and pancreatic cancer (FIG. 15), 2-FM was the most potent agent of those compared and the differences observed were especially large between 2-FM and its chloro and bromo derivatives. The differences were also significant when compared with 2-DG. The data, therefore, indicates the 2-FM may work differently than 2-DG and 2-FG in inhibiting tumor cell proliferation. The data further indicates that 2-FM can be a very effective antitumor therapeutic treatment for cancer, particularly brain and pancreatic tumors.

More particularly, FIG. 15 demonstrates the dose response curves of cell viability through MTT assays of Colo357 cell lines response to either treatment with 2-deoxy-glucose (2-DG), 2-fluoro-glucose (2-FG) or 2-fluoro-mannose (2-FM). As can be seen the shift of the dose response curves to the left indicating that 2-FM is more potent than either 2-DG or 2-FG. FIG. 16 demonstrates that halogen nature at 2-position of mannose is important factor affecting activity. The glioma cell line U251 MG was treated with either 2-chloro-mannose (2-CM), 2-bromo-mannose (2-BM), or 2-fluoro-mannose (2-FM). Again cell viability was measured by MTT assay and the result clearly shows superior activity of 2-FM when compared to the other Halogen based analogs. FIG. 17 demonstrates MTT assays of U87 cell line being treated with 2-fluoro-mannose (2-FM) in the presence of hypoxia (<1% oxygen) or normoxia (20% oxygen). As can be seen, data represents an unusual situation with this agent in U87 cell line is not more sensitive in the presence of hypoxia. This potentially indicates that an alternate mechanism of action for 2-FM may be responsible for the cell killing effect.

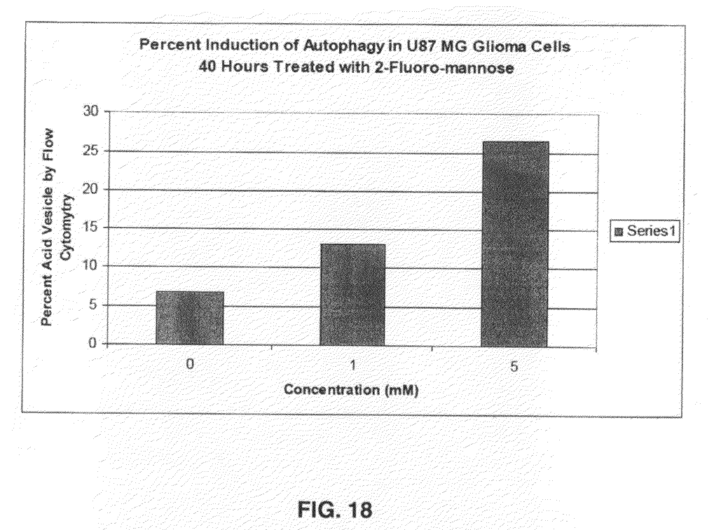

FIG. 18 demonstrates a unique and previously unidentified mechanism of 2-fluoro-mannose (2-FM). In U87 MG glioma cell lines 2-FM induces of cellular death through autophagy. Graphically represented are the results of flow cytometric analysis of Acidic Vesicular Organelles (AVO) by staining with acridine orange (see procedures), which is specific and characteristic of the autophagic process. The results indicate an increase of the percentage of cells undergoing autophagy with increasing dose exposure of 2-FM. This degree of induction of autophagy is impressive since the exposure time of only 40 hours is quite short to see this effect.

2-DG is currently being administered in a clinical trial to evaluate the extent to which the addition of a glycolytic inhibitor, which kills slow-growing hypoxic tumor cells, the most resistant cell population found in solid tumors, can increase treatment efficacy of standard chemotherapy targeting rapidly-dividing normoxic cells. The present invention arose in part from the discovery that, even in the presence of oxygen, certain tumor cell lines are killed when with 2-DG or 2-FM but not 2-deoxy-2-fluoro-D-glucose (2-FG) is administered. Because 2-FG and 2-DG both inhibit glycolysis, a mechanism other than blockage of glycolysis was presumed responsible for this effect.

Studies conducted in the 1970′s led to reports that 2-DG and 2-FM interfere with N-linked glycosylation of viral coat glycoproteins, which interference can be reversed by the addition of mannose. Because the difference between mannose and glucose lies in the orientation of the hydrogen at the 2-carbon position, and because 2-DG has two hydrogens at the 2-position (instead of a hydrogen and a hydroxyl group, as is the case for both mannose and glucose, 2-DG can be viewed either as a mannose or a glucose analog. Accordingly, 2-DG may act on both glycolysis and glycosylation.

The present invention provides methods to inhibit tumor cell proliferation regardless of whether the cells are in a hypoxic or normoxic environment, using hexose derivatives alone, or in combination with other anti-tumor treatments, including but not limited to cytotoxic agents that target normoxic cells, anti-angiogenic agents, radiation therapy, and surgery. The present invention also provides a basis for the clinical use of analogs such as 2-DG, 2-CM, and 2-FM as cytotoxic agents that can target both normoxic (via interference with glycosylation) and all hypoxic (via blockage of glycolysis) cancer cell populations in certain tumors types.

The examples below provide data that verify the effectiveness of the invention and confirms that 2-DG, 2-CM, and 2-FM, but not 2-FG, are toxic to select tumor cell types growing under normoxia. Some of the experiments described in the examples were designed to determine whether interference with glycosylation as opposed to inhibition of glycolysis is the mechanism responsible for the normoxic effect. While not wishing to be bound by theory, the results obtained support that these compounds can inhibit glycosylation and thereby kill certain cancer cell types independently of whether those cells are in a hypoxic environment.

The results are also supportive of the conclusion that 2-FM, 2-DG and 2-CM but not 2-FG disrupt the assembly of lipid-linked oligosaccharide chains and induce an unfolded protein response (UPR), which can be an indicator of interference with glycoprotein synthesis. In turn, the UPR leads to activation of UPR-specific apoptotic signals in sensitive but not resistant cells.

Tumor cell types that are sensitive to 2DG, 2-FM, and 2-CM under normoxic conditions have been identified. Cells were isolated from a tumor and tested ex vivo to determine if the cells are sensitive to 2-DG, 2-CM, or 2-FM under normoxic conditions. The examples below illustrate methods for determining whether a cell is sensitive. In other embodiments, molecular signatures of closely related 2-DG sensitive and resistant cell pairs are compared to a test cell line. Differences in the level and/or activity of phosphomannose isomerase and other enzymes involved in glycosylation and enzymes involved in 2-DG accumulation are described.

In the presence of oxygen (normoxic conditions), 2-DG is toxic to a subset of tumor cell lines. This result was surprising, because previous research demonstrated that tumor and normal cells are growth inhibited but not killed when treated with 2-DG under normoxia. This prior observation of growth inhibition was believed to be due to the accumulation of 2-DG to levels high enough to block glycolysis in cells under normoxia so that growth was reduced because of reduction in the levels of the intermediates of the glycolytic pathway, which are used for various anabolic processes involved with cell proliferation. The cells do not die, however, because if mitochondrial function is normal, then aerobically treated cells can survive blockage of glycolysis by 2-DG. One possible explanation for how a cell could be sensitive to 2-DG under normoxic conditions is, therefore, that the cell has defective mitochondria. In this regard, it is known that tumor cells utilize glucose through anaerobic glycolysis for the production of energy (ATP) instead of oxidative phosphorylation due to defective mitochondrial respiration. However, further experiments have demonstrated that other inhibitors of glycolysis, such as oxamate and 2-FG, are not toxic to these cells, so a defect in mitochondrial respiration is unlikely to account for their sensitivity to 2-DG. It was therefore hypothesized that a mechanism other than blockage of glycolysis is responsible for 2-DG toxicity in these select cell lines under normoxic conditions.

Accordingly, other hypotheses may explain the mechanism of this normoxic cytotoxicity. One potential mechanism was interference with glycosylation. Support for this potential mechanism could be identified in a series of papers from the late 1970′s in which it was reported that, in certain viruses, N-linked glycoprotein synthesis was inhibited by a number of sugar analogs, including 2-DG.

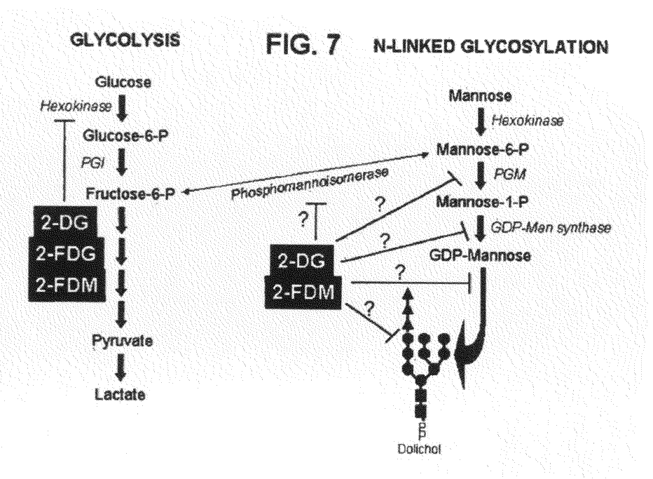

Glucose is metabolized through three major pathways: glycolysis, pentose phosphate shunt and glycosylation. FIG. 7 is a scheme diagram of the glycolysis and glycosylation metabolic pathways. After glucose enters the cytoplasm, hexokinase phosphorylates carbon 6 of glucose, resulting in synthesis of glucose-6-phosphate (G6P). If G6P is converted to fructose-6-phosphate by phosphoglucose isomerase (PGI), it can continue on the glycolysis pathway and produce ATP and pyruvate. Alternatively, G6P can also be used for synthesis of various sugar moieties, including mannose, which is required for assembly of lipid-linked oligosaccharides, the synthesis of which is performed in the ER. 2DG has been shown to interfere with two of the three metabolic pathways: it can block glycolysis by inhibiting PGI or it can disrupt the assembly of N-linked oligosaccharide precursor by interfering with the transfer of guanosine diphosphate (GDP) dolichol phosphate linked mannoses onto the N-acetylglucosamine residues and can deplete dolichol-P, which is required to transfer mannose from the cytoplasm to the lumen of the ER.

As noted above, because 2-DG has hydrogens at both positions of carbon 2 and is similar to a mannose analog. In contrast, the presence of a fluoride at this position in fluoro analogs creates a new enantiomeric center, and so the fluoro derivatives can only be considered analogs of either glucose or mannose; in depicting these analogs, the fluoride moiety is drawn “up” or above the plane of the carbohydrate ring for mannose analogs, and down for the glucose analogs.

For mannose to be added to a lipid-linked oligosaccharide chain, it must first be activated by being transferred to guanosine diphosphate (GDP) or dolichol phosphate. 2-DG undergoes conversion to 2-DG-GDP, which competes with mannose-GDP for the addition of mannose onto N-acetylglucosamine residues during the assembly of lipid-linked oligosaccharides. Thus, the aberrant oligo-saccharides produced as a result of 2-DG treatment resulted in decreased synthesis of the viral glycoproteins in the experiments reported in the scientific literature. In these experiments, the inhibitory effect of 2-DG was reversed with addition of exogenous mannose but not when glucose was added, further confirming that 2-DG acts somewhat like a mannose analog. These investigators also showed that another mannose analog, 2-fluoro-mannose (2-FM), had similar effects as 2-DG that were also reversed by mannose, indicating that the mannose configuration of these analogs may be important for their interference with glycosylation.

In addition, genetic studies have shown that disruption of glycosylation can have profound biological effects. The enzyme phosphomannose isomerase (PMI) is absent in patients suffering from Carbohydrate-Deficient Glycoprotein Syndrome Type 1b. The absence of this enzyme results in hypoglycosylation of serum glyco-proteins, leading to thrombosis and gastrointestinal disorders characterized by protein-losing enteropathy. When exogenous mannose is added to the diets of these patients, their symptoms disappeared, their serum glycoproteins returned to normal, and they recovered from the disease. This observation is consistent with a mechanism of action for the compounds useful in the present invention, as experimental data show that exogenous mannose can rescue the selected tumor cells that are killed when treated with 2-DG in the presence of normal oxygen levels. It is possible that these particular tumor cells are either down-regulating PMI or have a defect in this enzyme. On the other hand, enzymes that produce mannose intermediates necessary for N-linked glycosylation may be up-regulated in these cells, resulting in a higher 2-DG-GDP to mannose-GDP ratio and thereby causing this unusual sensitivity to 2-DG under normal oxygen conditions.

Regardless of mechanism, the present invention provides methods for treating cancer by administering 2-DG and other glucose and mannose analogs as single agents for treating tumors even under normoxic conditions. The compounds have been demonstrated to be effective against a number of tumor cells lines, including human breast (SKBR3), non small cell lung (NSCLC), gliomas, pancreatic and osteosarcoma cancer cell lines, all of which undergo cell death when treated with relatively low doses of 2-DG.

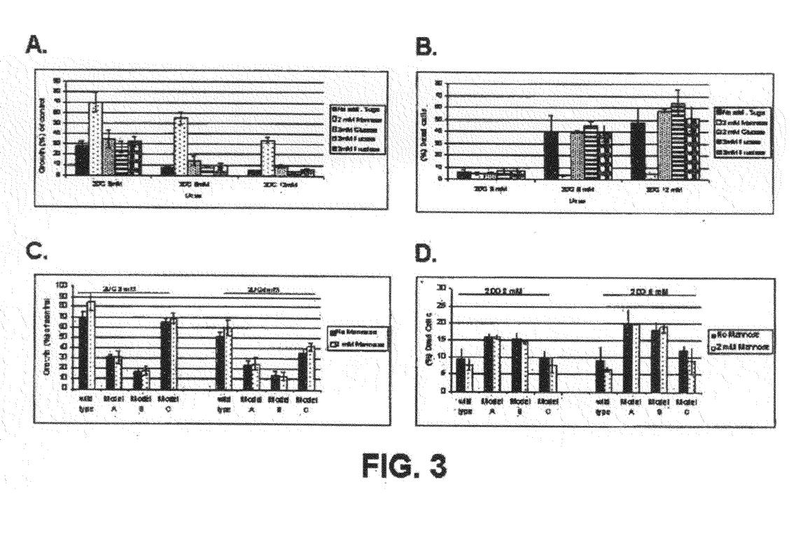

FIG. 3B is a chart showing the response of SKBR3 cells treated for 72 hrs with 2-DG, 2-FM and other agents under normal oxygen conditions at the doses indicated. Cytotoxicity was measured by trypan blue exclusion. The results show that 2-DG and the mannose analog 2-FM are toxic, while 2-FG, a glucose analog is not. Moreover, oxamate, an analog of pyruvate that blocks glycolysis at the lactic dehydrogenase level, is also not toxic to these cells growing under normoxic conditions. In contrast, the mannose analog, 2-FM also proved to be toxic in these cells, again indicating that a mannose backbone was important for compounds having this activity.

The inhibitory effect of 2-DG was reversed with addition of exogenous mannose but not when glucose was added, further confirming that 2-DG is acting as a mannose analog. Other testing showed that 2-DG is also toxic to a NSCLC growing under normoxic conditions and that addition of 1mM mannose reverses this toxicity.

This data further supports that 2-DG and 2-FM are toxic to select tumor cells growing under normoxic conditions due to interference with glycosylation. Additional evidence that these mannose analogs are working through this mechanism and not thru blockage of glycolysis is that the unfolded protein response proteins, GRP 78 and 94, indicative of mis-folded and or mis-glycosylated proteins are up-regulated by 2-DG and 2-FM in a dose-dependent manner but not by 2-FG; this effect is likewise reversed by addition of mannose.

Thus, the mannose analogs 2-DG and 2-FM, but not the glucose analog 2-FO, are toxic to select tumor cell types growing under normoxia, and the addition of mannose reverses this toxicity. Because 2-FG inhibits glycolysis better than 2-DG, interference with glycosylation and not inhibition of glycolysis is the mechanism believed to be responsible for this effect. As mentioned above, it has been reported that 2-DG interferes with N-linked glycosylation of viral coat proteins and that exogenously added mannose reverses the effect. The toxic effects of 2-DG on SKBR3, NSCLC and two other human tumor cell lines under normoxia are therefore likely to be due to interference of glycosylation. If this mechanistic theory is correct, then addition of mannose should reverse the toxicity of 2DG in these cell lines. Indeed, 1 mM of mannose reverses the toxic effects of 6 mM of 2DG in one of the cell lines tested (NSCLC).

Because blood levels of mannose are known to range between 50 and 60 micro g/ml, dose-response experiments to determine the minimal mannose dose necessary to reverse 2-DG toxicity can be performed. For example, this can be achieved by experiments in which growth medium is supplemented with dialyzed fetal bovine serum (FBS), because FBS normally contains residual amounts of mannose. Moreover, to confirm that the addition of mannose and not other sugars is required to reverse 2-DG toxicity, sugars known to participate in glycoprotein synthesis, i.e. glucose, fucose, galactose, and the like, can be tested for the ability to reverse 2-DG toxicity. If any of these sugars is able to reverse toxicity similarly, then their activity can be compared to that of mannose in the experiments described below for reversing the effects of 2-DG in inducing UPR and its consequences, interference with oligosaccharide chain elongation, and binding of conconavalin A. Overall, these experiments allow one to assess in vitro the dose of 2-DG or 2-FM that can be used in vivo to yield anti-tumor activity in the presence of physiologic concentrations of mannose. The therapeutically effective dose of orally administered 2-DG, 2-FM, and 2-CM for use in the methods of the invention will, however, typically be in the range of 5-500 mg/kg of patient weight, such as 50-250 mg/kg. In one embodiment, the dose is about 100 mg/kg of patient weight.

The present invention also provides a number of diagnostic methods a clinician can use to determine if a tumor or other cancer contains cells susceptible to the current method of treatment. In one embodiment, cells from a tumor are tested under normoxic conditions to determine if they are killed by 2-DG, 2-FM, or 2-CM. In another embodiment, this testing is conducted; then, mannose is added to determine if it reverses the cytotoxic effects.

In another embodiment, the test for susceptibility is performed using N-linked glycosylation as an indicator. As noted above, 2-DG and 2-FM but not 2-FG disrupt the assembly of lipid linked oligosaccharide chains, (2) induce an unfolded protein response (UPR), which is an indicator of interference with normal glycoprotein synthesis, and (3) activate UPR-specific apoptotic signals in 2-DG sensitive but not resistant cells. Additionally, mannose reverses these effects. Accordingly, these same tests can be performed on a tumor or cancer cell of interest to determine if that cell is susceptible to treatment with the present method.

As noted above, the incorporation of mannose into a lipid-linked oligosaccharide chain occurs on the cytoplasmic surface of the ER in virus-infected cells, and this incorporation can be inhibited by GDP derivatives of 2-DG or 2-FM, i.e. GDP-2DG and GDP-2-FM. Normally, after the fifth mannose has been added, the lipid-linked oligosaccharide chain flips to face the lumen of the ER. To continue adding mannose to the growing chain, dolichol-phosphate (Dol-P) is used as a carrier to transport mannose from the cytoplasm to the matrix of ER. 2-DG-GDP competes with mannose-GDP for binding to dolichol and thereby further interferes with N-linked glycosylation. Moreover, dolichol-linked 2-DG also competes with the transfer of mannose onto the oligosaccharide chain in the ER. Accordingly, experiments can be performed to demonstrate the effects of 2-DG and 2-FM on the formation of lipid linked oligosaccharide precursors and the derivatives of mannose, i.e. mannose-6-phosphate, mannose-1-phosphate, GDP-mannose and Dol-P-mannose in both 2-DG sensitive and resistant cell lines. This in turn demonstrates the step or steps in oligosaccharide assembly that are inhibited by 2-DG and 2-FM. This in turn allows one to characterize other cell types as sensitive or resistant based on the oligosaccharides produced (and not produced) upon exposure to 2-DG, 2-FM, and/or 2-CM.

Previously established chromatographic methods can be used to collect and measure the amount of mannose derivatives and lipid-linked oligosaccharide precursors in SKBR3 and NSCLC cells. Briefly, cells can be labeled with [2-H³] mannose and cell lysates extracted with chloroform/methanol (3:2) and chloroform/methanol/water (10:10:3) to collect Dol-P-Man and lipid linked oligosaccharides, respectively.

Aliquots containing Dol-P-Man can be subjected to thin layer chromatography while the lipid linked oligosaccharides can be separated by HPLC. Eluate fractions can be analyzed by liquid scintillation counting. Mannose phosphates and GDP-mannose can be separated by descending paper chromatography and [2-³H] mannose released from each fraction by mild acid hydrolysis and measured. The values derived from cells treated with 2-DG or 2-FM can be compared to untreated controls to demonstrate the effects of these drugs on N-linked oligosaccharide precursors and mannose derivatives. Because exogenous mannose reverses 2-DG toxicity, one can also test whether mannose also reverses the 2-DG glycosylation perturbations observed.

In addition to 2-DG and 2-FM, two other glycosylation inhibitors, tunicamycin and deoxymannojirimycin (DMJ), which can inhibit specific steps of N-linked glycosylation, can be used as positive controls. Tunicamycin interferes with the addition of the first N-acetylglucosamine residue onto dolichol pyrophosphate, and DMJ is a specific inhibitor of mannosidase I, which trims 3 mannose residues at the end of the N-linked oligo-saccharide chain. Thus, exogenous mannose should not be able to reverse either the toxicity or the effects on glycosylation of either of these agents. Moreover, because the glucose analog 2-FG does not kill SKBR3 and NSCLC cells under normoxia but is more potent than 2-DG in blocking glycolysis and killing hypoxic cells, it can interfere with glycolysis without affecting glycosylation and so can be used as a tool in such testing as well.

Interference with the process of N-linked glycosylation in the endoplasmic reticulumn (ER) causes improper folding of glycoproteins, which elicits an ER stress response called the unfolded protein response (UPR). Reminiscent of the P53 response to DNA damage, the ER responds to stress in much the same way by (1) increasing folding capacity through induction of resident chaperones (GRP 78 and GRP 94), (2) reducing its own biosynthetic load by shutting-down protein synthesis, and (3) incr easing degradation of unfolded proteins. If the stress cannot be alleviated, apoptotic pathways are initiated and the cell subsequently dies. Thus, one measurement of interference with glycosylation is upregulation of UPR.

When SKBR3 cells are treated with 2-DG, both of these ER stress response proteins, GRP 78 and 94, increase as a function of increasing dose; mannose reverses this induction. 2-FG does not induce these proteins. Accordingly, in another embodiment of this invention, this response is used to determine if a tumor or cancer cell is susceptible to treatment in accordance with the present method. Cell lines that are not sensitive to 2-DG under normoxic conditions can be similarly used as negative controls in which the absence of upregulation of these proteins correlates with their resistance to 2-DG.

When ER stress cannot be overcome, apoptotic signals are initiated. ER stress induces a mitochondrial dependent apoptotic pathway via CHOP/GADD153, a nuclear transcription factor that down-regulates BCL-2, and a mitochondrial independent pathway by caspases 4 and 5 in human and caspase 12 in mouse cell lines. Thus, experiments can be performed to determine whether the apoptotic signals particular to ER stress are activated in 2-DG sensitive but not resistant cells. Up-regulation of CHOP/GADD153 and activation of caspases 4 and 5 can be assayed by western blot. As with the previous tests, if this up-regulation is specific to 2-DG-sensitive lines, then the up-regulation observed in a test cancer cell serves as an indicator that the cancer from which the cell was derived is susceptible to treatment in accordance with the present invention.

Because SKBR3 abundantly expresses the glycoprotein ErbB2, it is expected that 2-DG would affect the N-linked glycosylation of this protein, leading to mis-folding and degradation. Western blots of ErbB2 from SKBR3 cells treated with 2-DG can be compared to those from untreated cells to determine the overall level of this protein. Furthermore, the mannose content of ErbB2 following 2-DG treatment can be analyzed by immuno-precipitating this protein and blotting with Conconavalin A, a lectin that recognizes high mannose type N-linked oligosaccharides. Because it is likely that mannose analogs can inhibit the mannose content of not only ErbB2, but all N-linked glycoproteins, whole cell lysates obtained from these cells can also be probed with this lectin. Ponceau stain, which binds to all proteins, can be used as a negative control to verify that 2DG and 2FM specifically affects glycoproteins, and again, this or similar methodology can be used to determine if a cancer or tumor cell is susceptible to treatment in accordance with the present invention.

Even if ER stress indicative of interference with N-linked glycosylation is indeed confirmed to occur by 2-DG and 2-FM, interference with O-glycosylation, which takes place in the cytoplasm as opposed to the ER, can also be evaluated. The scientific literature reports that 2-DG can inhibit the trimming of N-acetylglucosamine residues from an O-glycosylated transcription factor, Sp1, resulting in inhibition of binding to its respective promoters. Sp1 is an important transcription factor for activating numerous oncogenes, which if affected by 2-DG could, at least in part, explain why SKBR3 cells growing under normoxia are sensitive to 2-DG. Thus, the glycosylation pattern of Sp1 following treatment with 2-DG and 2-FM can be investigated by immunoprecipitating and probing with WGA, a lectin that specifically binds O-glycosylated proteins. To the extent that 2-DG affects Sp1 and O-linked glycosylation, this alteration of glycosylation can be measured and used as an indicator that a tumor or other cancer cell line is susceptible to 2-DG-mediated cell killing.

The cell death triggered by the unfolded protein response, which occurs in the endoplasmic reticulum of every cell in response to mis-folded proteins, can be enhanced by administration of an additional agent, versipelostatin. Thus, in one embodiment 2-DG, 2-FM, and/or 2-CM is administered to a patient in need of treatment for cancer, and versipelostatin is co-administered to said patient.

Similarly, the cell death that occurs in response to mis-folding of proteins can be enhanced by blocking the proteolysis of the misfolded glycoproteins with a proteosome inhibitor. Thus, in another embodiment, the invention provides a method of treating cancer by administering a proteosome inhibitor in combination with 2-DG, 2-FM, and/or 2-CM. In one embodiment, the proteosome inhibitor is Velcade.

Certain types of cancers may be more susceptible to treatment with the present method than others. To identify such types, one can examine a variety of cell types in accordance with the methods of the invention. For example, one can obtain a variety of cancer cell lines from the ATCC and screen them as described above to identify other cell types exquisitely sensitive to mannose analogs, such as 2DG and 2FM, in the presence of oxygen. Cells that are killed in concentrations of 5 mM 2-DG or 2-FM or less are identified as susceptible. These susceptible tumor cell lines can also be tested for their sensitivity to 2-FG and oxamate at doses up to 20 mM and 30 mM, respectively. If interference with glycosylation is the mode of toxicity of 2-DG and 2-FM, then these cell lines should be resistant to the other glycolytic inhibitors, 2-FG and oxamate, unless they have a deficiency in mitochondria oxidative phosphorylation. To confirm the mitochondria functionality of these cells, respiration can be measured using, for example, a Clark electrode apparatus. To confirm that toxicity of 2-DG and 2-FM is due to interference with glycosylation in these cell lines, recovery of the cell death by mannose can be assayed as described above.

The molecular basis for one cell being resistant to the current method and another not may be due to difference in the expression of the gene involved in the synthesis of GDP-mannose from glucose i.e. phosphoglucose isomerase (PMI), which converts glucose-6-phosphate to mannose-6-phosphate (see FIG. 7). A deletion in PMI, as mentioned above, was shown to cause glycosylation syndrome lb, which resulted in hypoglycosylation of serum glycoproteins leading to thrombosis and gastrointestinal disorders in a patient identified with this defect. Addition of mannose to the diet was shown to alleviate the patient's symptoms as well as normalize his glycoproteins. Thus, a deficiency or down-regulation of this enzyme could explain the toxicity of 2DG and 2FM and reversal by exogenous mannose in the sensitive cell lines so far tested.

The reason why down-regulation or deletion of PMI could lead to 2-DG toxicity in the sensitive cell lines is that, in the absence of this enzyme, cells are dependent on exogenous mannose (present in serum) to synthesize N-linked oligosaccharide precursors. Mannose concentrations in the serum of mammals (50-60 microg/ml), or in the medium used for in vitro studies, are known to be significantly less than the concentration of glucose. Thus, in cells with deleted or down-regulated PMI, low doses of 2-DG and 2-FM could favorably compete with the low amounts of mannose present in serum, resulting in complete blockage of the addition of this sugar onto the oligosaccharide chains. On the other hand, cells with normal PMI can produce GDP-mannose from glucose; thus, much higher doses of 2-DG or 2-FM are necessary to cause complete disruption of oligosaccharide assembly. This could explain why most cells tested are resistant to 2DG under normoxic conditions. Direct measurements of the activity of this enzyme can be used in accordance with the invention to determine whether defective or low PMI levels are responsible for the sensitivity to 2-DG and 2-FM in select cells growing under normoxia, and if so, then can be used to identify tumor and cancer cells susceptible to treatment in accordance with the present method. Another, but less likely, possibility to explain this unusual sensitivity, is that the PMI in these select cells is inhibited more by 2-DG and 2-FM than in the majority of normal and tumor cell lines that are unaffected by these agents when growing under normal oxygen tension. In order to test this directly, cell extracts can be isolated from SKBR resistant and sensitive cell pairs and the ability to convert glucose-6-P to mannose-6-P can be determined in the presence or absence of 2-DG and 2-FM.

If decreased PMI activity is not responsible for 2-DO toxicity in SKBR3 sensitive cells, then an alternative mechanism to explain this is up-regulation of genes that encode enzymes involved in the production of mannose derivatives used for oligosaccharide assembly, i.e. phosphomannomutase (PMM) and GDP-Man synthase (FIG. 7). The possibility exists that cells sensitive to 2-DG are undergoing increased glycosylation and therefore up-regulate either one or both of these enzymes. Such a cell would accumulate more 2-DG-GDP, therefore leading to greater interference with glycosylation and consequently cell death than a resistant cell in which glycosylation was occurring at a slower rate or capacity.

Regardless of whether up-regulation of glycosylation turns out to be a mechanism by which cells become sensitive to 2-DG, the total amount of 2-DG that is accumulated or incorporated into a cell also contributes to its increased sensitivity. Thus, uptake and accumulation studies using [³H] labeled 2-DG can be performed determine if a cell higher levels of glucose transporter, rendering it more susceptible to treatment in accordance with the present method.

One can obtain 2-DG resistant mutants from sensitive cells by treating the latter with increasing doses of 2-DG and selecting for survival. Resistant mutants and their parental sensitive counterparts can be used in the methods described. Such studies should also provide a means of understanding mechanisms by which cells become resistant to 2-DG and therefore may be applicable to better use of this drug clinically. The foregoing discussion reflects that a molecular signature can be used to predict which tumor cell types will be sensitive to 2-DO and 2-FM in the presence of oxygen.

Execution of cell death shows a remarkable plasticity spanning the range between apoptosis and necrosis. Using established methods to compare the mode of cell death by investigating the type of DNA cleavage, changes in membrane composition, integrity, and tone can determine the mechanisms of cell death induced by interference with glycosylation and by inhibition of glycolysis. Inhibition of both glycolysis and oxidative phosphorylation results in severe ATP depletion, thereby causing a switch from apoptosis to necrosis. Because ATP is required to activate caspases, when it is severely depleted, apoptosis is blocked, and eventually, without energy, the cell succumbs via necrosis. An aerobic cell treated with a glycolytic inhibitor is able to produce ATP via oxidative phosphorylation fueled by either amino acids and or fats as energy sources. Thus, when 2-DG induces a UPR response leading to cell death under normoxia, it is believed that cells will undergo apoptosis. Conversely, in hypoxic cell models, it is expected that when the dose of 2-DG is high enough to block glycolysis, these cells should undergo ATP depletion and die through necrosis.

One can therefore use established methods of assaying for apoptosis and necrosis and determine whether 2-DG is killing cells via apoptosis, necrosis and or a mixture of both. Several apoptotic parameters can be assayed to distinguish necrosis from apoptosis by using flow cytometry analysis. Following 2-DG treatment, cells can be dual-stained with Annexin-V and propidium iodide to detect exposure of phosphoatidyl serine on the cell surface and loss of cell membrane integrity, respectively. Staining with either annexin-V alone or both annexin-V and propidium iodide indicates apoptosis, while staining with propidium iodide alone indicates necrosis. Furthermore, two of the fmal outcomes of apoptosis, nuclear DNA fractionation and formation of single stranded DNA, can also be measured. These two latter parameters have been reported to be unique to apoptotic cell death and have been used by various investigators to differentiate apoptosis from necrosis. ATP levels can also be assayed to determine whether they correlate with the modes of death detected.

Moreover, if 2-DG induces both apoptosis and necrosis in hypoxic cells, then one can determine the mode of cell death induced by 2-FG under hypoxic conditions. As mentioned above, 2-FG does not interfere with glycosylation and is a more potent glycolytic inhibitor than 2-DG. Thus, it is expected that the cell death induced by 2-FG will occur solely via necrosis.

Cell lines proven to be sensitive to 2DG and/or 2FM and/or 2-CM in vitro under normoxia that grow readily in nude mice can be used to demonstrate that 2DG (and 2-FM and 2-CM) is effective as a single agent against them when given in vivo. After tumors reach a certain size, treatment with 2DG will be applied via intraperitoneal injection. Dose and treatment regimen of 2DG according to the minimal lethal dose established previously in these animals can be used to demonstrate tumor regression and cytotoxicity.

### Example 1

### Materials and Methods

Isolation of resistant Mutants. 2-DG sensitive SKBR3 and NSCLC cells are exposed to increasing doses of 2-DG and resistant colonies are isolated and cloned at the appropriate doses of 2-DG. The cloned 2-DG resistant cells are then analyzed and compared to the wild-type sensitive counterpart for expression of specific genes that may be responsible for this unique sensitivity.

Drugs and Antibodies. Rho 123, oligomycin, staurosporin, and 2-DG, 2-FG, 2-FM, tunicamycin, deoxymannojirinomycin are obtained from Sigma Chemical Co. The following primary Abs can be used: monoclonals to HIF-1a and LDH-a. (BD Biosciences); erbB2 (Calbiochem, USA); Grps 78 & 94, (StressGen, USA); caspases 4 and 5 (StressGen, USA); and actin (Sigma Chemical Co.); polyclonal abs to GLUT-1 (USA Biological) and GADD153/CHOP (Santa Cruz, USA). The secondary antibodies are horseradish peroxidase conjugated rabbit anti-mouse and goat anti-rabbit (Promega,Co.).

Cytotoxicity assay and Rapid DNA Content Analysis. Cells are incubated for 24 hr at 37 degrees C. in 5% CO₂ at which time drug treatments begin and are continued for 72 hr. At this time, attached cells are trypsinized and combined with their respective culture media followed by centrifugation at 400 g for 5 min. Pellets containing the cells are either resuspended in 1.5 ml of a medium/trypan blue mixture for cytotoxicity assays or propidium iodide/hypotonic citrate staining solution for determining the nuclear DNA content and cell cycle by a Coulter XL flow cytometer. A minimum of 10,000 cells are analyzed to generate a DNA distribution histogram.

Lactic acid assay. Lactic acid is measured by adding 0.025 ml of deproteinated medium, from treated or non-treated cultures, to a reaction mixture containing 0.1 ml of lactic acid dehydrogenase (1000 units/ml), 2 ml of glycine buffer (glycine, 0.6 mol/L, and hydrazine, pH 9.2), and 1.66 mg/ml NAD. Deproteinization occurs by treating 0.5 ml of medium from test cultures with 1 ml of perchloric acid at 8% w/v, vortexing for 30 s, then incubating this mixture at 4 degrees C. for 5 min, and centrifuging at 1500 g for 10 min. The supernate is centrifuged three times more, and 0.025 ml of a final clear supernate is used for lactic acid determinations. Formation of NADH is measured with a Beckman DU r 520 UV/vis spectrophotometer at 340 nm, which directly corresponds to lactic acid levels as determined by a lactate standard curve.

2-DG Uptake. Cells are seeded into Petri dishes, and incubated for 241u^{.} at 37 degrees C. and 5% CO₂. The medium is then removed and the plates are washed with glucose- and serum-free medium. 2 ml of serum-free medium containing ³H labeled 2-DG are added to the dish (1 ICi/plate), and the plates are incubated for the appropriate amount of time. The medium is then removed, the plates are washed three times with at 4 degrees C., and serum-free medium containing 100 micro M of unlabeled 2-DG, and 0.5 ml of 1N NaOH is added. After incubating at 37 degrees C. for 3 hr (or overnight), the cells are scraped and homogenized by ultrasonication (10 seconds). The solution is collected into tubes for ³H quantification (saving a portion for protein assay). 100 micro L of formic acid, 250 micro L of sample, and 7. ml of scintillation cocktail are combined in a ³H counting vial, and read with a scintillation counter. Transport rate (nmol/mg protein/time) is calculated by Total CPM/Specific Radioactivity/Total Protein.

ATP quantitation assay. The ATP lite kit (Perkin Elmer) can be used to quantify levels of ATP. About 50 micro L of cell lysis solution are added to 100 micro L of cell suspension in a white-bottom 96-well plate. The plate is incubated at room temperature on a shaker (700 rpm) for five minutes. 50 micro L of substrate solution is then added to the wells and shaken (700 rpm) for another five minutes at room temperature. The plate is then dark adapted for ten minutes and measured for luminescence.

Metabolic labeling and extraction of Dol-P Man and lipid linked oligosaccharides (LLO). According to the procedure described by Lehle, cells are labeled with [2-³H] mannose for 30 min, scraped into 2 ml of ice-cold methanol and lysed by sonification. After adding 4 ml of chloroform, the material is sonified, followed by centrifugation for 10 min at 5000 rpm at 4 degrees C. Supernatants are collected and the pellets extracted twice with chloroform/methanol (3:2) (C/M). The combined supernatants containing Dol-P-Man and lipid linked oligosaccharides of small size are dried under N₂, dissolved in 3 ml of C/M, washed, and analyzed by thin layer chromatography on Silica gel 60 aluminium sheets in a running buffer containing C/M/H₂O (65:25:4). The remaining pellet containing the large size LLOs is washed and extracted with C/M/H₂O (10:10:3). Corresponding aliquots of the C/M and C/M/H₂O extracts are combined and dried under N₂ and resuspended in 35 Υ1 1-propanol. To release the oligosaccharides by mild acid hydrolysis, 500 ΥI 0.02 N HCI are added followed by an incubation for 30 min at 100 degrees C.

The hydrolyzed material is dried under N₂ and then resuspended by sonification in 200 Υ1 of water and cleared by centrifugation. The supernatant containing the released oligosaccharides are used for HPLC analysis.

Size fractionation of oligosaccharides by HPLC. The separation of LLOs can be performed on a Supelcosil LC-NH₂ column (25 cm×4.6 mm; 5 Υm; Supelco) including a LC-NH₂ (2 cm×4.6 mm) precolumn. A linear gradient of acenotrile from 70% to 50% in water is applied at a flow rate of 1 ml/min. Eluate fractions are analyzed by liquid scintillation counting.

Preparation of mannose 6-phosphate, mannose 1-phosphate, GDP-mannose. After labeling with [2-³H] mannose, cells are harvested and free mannose is separated from nucleotide linked and phosphorylated mannose derivatives by paper chromatography as described by Korner et al. Eluate fractions are analyzed by liquid scintillation counting.

Western Blot analysis. Cells are plated at 10⁴ cell cm⁻² and grown under drug treatment for the indicated times. At the end of the treatment period, cells are collected and lysed with RIPA buffer (150 mM NaCl, 1% Np-40, 0.5% DOC, 0.1%SDS, 50 mM Tris-HCI, ph 8.0) supplemented with a proteinase inhibitor cocktail. DNA is fragmented by passing the solution through a 21 G needle 10 times. Protein concentrations are measured by a Super Protein Assay kit (Cytoskeleton, USA). Samples are mixed with 2× Laemmli sample buffer (Bio-Rad, USA) and run on a SDS-polyacrylamide gel. Gels are transferred to nitrocellulose membranes (Amersham, USA) and probed with specific antibodies. Following probing, membranes are washed and incubated with an HRP conjugated secondary antibody. Chemiluminesence is detected by exposure to film.

Where indicated, membranes are stripped with Stripping Buffer (Pierce, USA) and reprobed with anti-actin primary antibody.

Immunoprecipitation of ErbB2. Following treatment of cells for 24 hours, they are lysed by RIPA (15 mM NaCl, 1% Np-40, 0.1% SDS) and sonicated. Cell lysates are incubated with CnBr activated Sepharose beads (Amersham, USA) linked to monoclonal ErbB2 antibody (Calbiochem, USA) and spun at 400 g for 5 min.

Immunoprecipitated ErbB2 is loaded onto SDS-PAGE gels and blotted with Conconavalin A, which binds specifically to mannose residues of glycoproteins.

Apotosis Assay. The apoptosis ELISA assay is used as described and is based on selective DNA denaturation in condensed chromatin of the apoptotic cells by formamide and reactivity of single-stranded DNA (ssDNA) in apoptotic cells with monoclonal antibodies highly specific to ssDNA. These antibodies specifically detect apoptotic cells and do not react with the necrotic cells.

Investigation of cell death mechanism by flow cytometry. Apoptosis is distinguished from necrosis by An-nexin-V-Fluos Staining kit (Roche, USA). Following indicated treatments,10⁶ cells are resuspended in incubation buffer containing FITC conjugated Annexin-V and propidium iodide to detect phosphotdylserine and plasma membrane integrity, respectively. After incubation, cells are analyzed by a flow cytometer using 488 nm excitation and a 515 nm bandpass filter for fluoroscein detection and a filter >600 nm for PI detection.

Gene expression profiling. A Gene-array kit can be purchased from Super Array Inc. Total RNA from selected cell-lines is probed with dCTP [α-³²P] (3000 Ci/mmol) through a reverse transcription reaction. The labeled cDNA probed is then added to pre-hybridized array membrane and incubated in a hybridization oven overnight. After multiple washings to remove free probe, the membrane is exposed to X-ray film to record the image.

In vivo tumor experiments. The protocol described for 2-DG+Dox reported in Cancer Res. 2004 (by Lampidis et al.) can be replicated substituting 2-FG for 2-DG. Nude mice, strain CD1, 5 to 6 weeks of age, weighing 30 g, are implanted (S.C.) with 100 11 of human osteosarcoma cell line 143b at 10⁷ cells/ml. When tumors are 50 mm³ in size (9-10 days later), the animals are pair-matched into four groups (8 mice/group) as follows: saline-treated control; 2-FG alone; Dox alone; and Dox+2-FG. At day 0, the 2-FG alone and Dox+2-FG groups receive 0.2 ml of 2-FG i.p. at 75 mg/ml (500 mg/kg), which is repeated 3× per week for the duration of the experiment. On day 1, the Dox and Dox+2-DG groups receive 0.3 ml of Dox i.v. at 0.6 mg/ml (6 mg/kg), which is repeated once per week for a total of three treatments (18 mg/kg). Mice are weighed, and tumor measurements are taken by caliper three times weekly.

SKBR3 cells are implanted and tested in the above model with 2-DG or 2-FM without doxorubicin (Dox).

### Example 2

### Normoxic Sensitivity of Certain Tumor Cells to Mannose Derivatives

Cells growing under hypoxia are solely dependent on glucose metabolism via glycolysis for energy production. Consequently, when this pathway is blocked, with 2-deoxy-D-glucose (2-DG), hypoxic cells die. In contrast, when glycolysis is blocked under normoxia most cells survive, because fats and proteins can substitute as energy sources to fuel mitochondrial oxidative phosphorylation. The present invention is based in part on the discovery that, under normal oxygen tension, a select number of tumor cell lines are killed at a relatively low dose of 2-DG (4 mM). It has been shown previously that 2-DG interferes with the process of N-linked glycosylation in viral coat glycoprotein synthesis, which can be reversed by addition of exogenous mannose. Because the 2-DG toxicity under normoxia described herein can be completely reversed by low dose mannose (2 mM), glycosylation and not glycolysis is believed to be the mechanism responsible for these results. Additionally, 2-fluoro-deoxy-D-glucose (2-FDG), which is more potent than 2-DG in blocking glycolysis and killing hypoxic cells, shows no toxicity to any of the cell types that are sensitive to 2-DG under normoxic conditions.

To investigate the effect of 2-DG on glycoprotein synthesis, concanavalin A (which specifically binds to mannose moieties on glycoproteins) was used in studies that showed that 2-DG but not 2-FDG decreased binding, which was reversible by addition of exogenous mannose. Similarly, the unfolded protein response (UPR) proteins, grp 98 and 78, which are known to be induced when n-linked glycosylation is altered, were found to be upregulated by 2-DG but not 2-FDG, and again, this effect could be reversed by mannose. Moreover, 2-DG induces cell death via upregulation of a UPR specific transcription factor (GADD154/CHOP), which mediates apoptosis. Thus, in certain tumor cell types, 2-DG can be used clinically as a single agent to kill selectively both the aerobic (via interference with glycosylation) as well as the hypoxic (via inhibition of glycolysis) cells of a solid tumor.

Despite angiogenesis, the metabolic demands of rapid tumor growth often outstrip the oxygen supply, which contributes to formation of hypoxic regions within most solid tumors. The decrease in oxygen levels that occurs as the tumor grows, leads to slowing the replication rate of cells in the hypoxic portions, resulting in resistance to most chemotherapeutic agents which normally target rapidly proliferating cells. Brown, J. M, et al., Exploiting Tumor Hypoxia In Cancer Treatment, Nat Rev Cancer 2004; 4:437-47. Hypoxic cells are also resistant to radiation treatment due to slow growth and the absence of oxygen necessary to produce reactive oxygen species. Semenza, G. L., Intratumoral Hypoxia, Radiation Resistance, And HIF-1, Cancer Cell 2004; 5:405-406. In addition to these disadvantages for cancer treatment, hypoxia renders a tumor cell dependent on glycolysis for energy production and survival. Under hypoxia, oxidative phosphorylation, the most efficient means of ATP production, is inhibited, leaving glycolysis as the only means for producing ATP. Thus, blocking glycolysis in hypoxic tumor cells should lead to cell death. Indeed, under 3 different conditions of simulated hypoxia in vitro, it has been shown that tumor cells can be killed by inhibitors of glycolysis. Maher, J. C., et al., Greater Cell Cycle Inhibition And Cytotoxicity Induced By 2-Deoxy-D-Glucose In Tumor Cells Treated Under Hypoxic vs Aerobic Conditions, Cancer Chemother Pharmacol 2004; 53:116-122. Moreover, inhibition of glycolysis in normally oxygenated cells does not significantly affect their energy production, because alternative carbon sources, i.e. amino acids and fats, can be utilized to drive mitochondrial oxidative phosphorylation. Therefore, glycolytic inhibitors can be used to target hypoxic tumor cells selectively, without showing much toxicity to normal or tumor cells growing aerobically. Boros, L. G., et al., Inhibition Of Oxidative And Nonoxidative Pentose Phosphate Pathways By Somatostatin: A Possible Mechanism Of Antitumor Action, Med Hypotheses 1998; 50:501; LaManna, J. C., Nutrient Consumption And Metabolic Perturbation, Neurosurg Clin N Am 1997; 8:145-163.

In fact, in vivo experiments have shown that 2-DG (targeting slow-growing hypoxic tumor cells) increases the efficacy of standard chemotherapeutic agents (directed against rapidly proliferating aerobic cells) in different human tumor xenografts. Maschek, G., et al., 2-Deoxy-D-Glucose Increases The Efficacy Of Adriamycin And Paclitaxel In Human Osteosarcoma And Non-Small Cell Lung Cancers In Vivo, Cancer Res 2004; 64:31-4. The results of these studies as well as data from in vitro models of hypoxia has led to testing this strategy for improving chemotherapy protocols in humans in the form of a Phase I clinical trial entitled “A Phase I dose escalation trial of 2-deoxy-D-glucose alone and in combination with docetaxel in subjects, with advanced solid malignancies, “which is currently ongoing. Maher, J. C., et al., Greater Cell Cycle Inhibition And Cytotoxicity Induced By 2-Deoxy-D-Glucose In Tumor Cells Treated Under Hypoxic vs Aerobic Conditions, Cancer Chemother Pharmacol 2004; 53:116-122. The data from animal studies, as well as the preliminary results from the Phase I clinical trial, indicate that 2-DG is well-tolerated and relatively non-toxic to normal cells.

Although theoretically tumor cells with mitochondria able to undergo oxidative phosphorylation should not be killed by the glycolytic inhibitor 2-DG, a select number of cancer cell lines die in the presence of oxygen with low doses of this sugar analog. The mechanism of toxicity is not via blockage of glycolysis, because these cell lines undergo normal mitochondrial respiration and are resistant to other glycolytic inhibitors. A similar mechanism has been shown in viral glycoprotein synthesis, in which 2-DG blocks N-linked glycosylation by interfering with lipid linked oligosaccharide assembly. Datema, R., et al., Interference With Glycosylation Of Glycoproteins, Biochem J 1979;184: 113-123; Datema, R., et al., Formation Of 2-Deoxyglucose-Containing Lipid-Linked Oligosaccharides, Eur J Biochem 1978;90: 505-516. The toxicity with 2-DG in the select tumor cell lines growing under normoxia appears to be due to a similar mechanism.

In accordance with the present invention, 2-DG can be used as a single agent in certain patients with solid tumors containing cells sensitive to 2-DG under normoxia. Thus, in these patients 2-DG should have a dual effect by (1) targeting the aerobic tumor cell population via interference with glycosylation; and (2) inhibiting glycolysis in the hypoxic portion of the tumor; both mechanisms lead to cell death.

Materials and Methods

Cell Types

The p⁰ cells were isolated by treating osteosarcoma cell line 143B (wt) with ethidium bromide for prolonged periods, as previously described. King, M. P., et al., Human Cells Lacking Mtdna: Repopulation With Exogenous Mitochondria By Complementation, Science 1989; 246: 500-503. Because the p⁰ are uridine and pyruvate auxotrophs, they are grown in DMEM (GIBCO, USA) supplemented with 10% fetal calf serum, 50 micro g/ml of uridine and 100 mM sodium pyruvate. The SKBR3 cell line was obtained from Dr. Joseph Rosenblatt's laboratory at the University of Miami. The pancreatic cancer cell lines 1420 and 1469, the ovarian cancer cell line SKOV3, the cervical cancer cell line HELA, and the osteosarcoma cell line 143B were purchased from ATCC. The non-small cell lung cancer and small cell lung cancer cell lines were derived from patients by Dr. Niramol Savaraj at the University of Miami. SKBR3 and SKOV3 cells were grown in McCoy's 5A medium; 1420, 1469 and 143B were grown in DMEM (GIBCO, USA); and HELA was grown in MEM (GIBCO, USA). The media were supplemented with 10% fetal bovine serum. All cells were grown under 5% CO₂ and 37° C.

Drugs and Chemicals

2-DG, oligomycin and tunicamycin were purchased from Sigma. 2-FDG and 2-FDM were a kind gift of Dr. Priebe (MD Anderson Cancer Center, TX).

Hypoxia

For studies in hypoxic conditions (Model C), cells are seeded and incubated for 24 hr at 37° C. and 5% CO2 as described below for direct cytotoxicity assays. After the 24 hr incubation, cells receive drug treatment and are placed in a Pro-Ox in vitro chamber attached to a model 110 oxygen controller (Reming Bioinstruments Co. Redfield, N.Y.) in which a mixture of 95% Nitrogen and 5% CO2 is used to perfuse the chamber to achieve the desired O₂ levels (0.1%).

Cytotoxicity Assay

Cells are incubated for 24 hr at 37° C. in 5% CO₂ at which time drug treatments begin and are continued for 72 hr. At this time, attached cells are trypsinized and combined with their respective culture media followed by centrifugation at 400 g for 5 min. The pellets were resuspended in 1 ml of Hanks solution and analyzed by Vi-Cell (Beckman Coulter, USA) cell viability analyzer.

Lactic Acid Assay

Lactic acid is measured by adding 0.025 ml of deproteinated medium, from treated or non-treated cultures, to a reaction mixture containing 0.1 ml of lactic dehydrogenase (1000 units/ml), 2 ml of glycine buffer (glycine, 0.6 mol/L, and hydrazine, pH 9.2), and 1.66 mg/ml NAD. Deproteinization occurs by treating 0.5 ml of medium from test cultures with 1 ml of perchloric acid at 8% w/v, vortexing for 30 s, then exposing this mixture to 4 degrees C. for 5 min, and centrifugation at 1500 g for 10 min. The supernatant is centrifuged three times more, and 0.025 ml of a final clear supernatant are used for lactic acid determinations as above. Formation of NADH is measured with a Beckman DU r 520 UV/vis spectrophotometer at 340 nm, which directly corresponds to lactic acid levels as determined by a lactate standard curve.

ATP Quantification Assay

The ATP lite kit (Perkin Elmer) can be used to quantify levels of ATP. About 50 ml of cell lysis solution are added to 100 ml of cell suspension in a white-bottom 96-well plate. The plate is incubated at room temperature on a shaker (700 rpm) for five minutes. About 50 ml of substrate solution are then added to the wells and shaken (700 rpm) for another five minutes at room temperature. The plate is then dark adapted for ten minutes and measured for luminescence.

Western Blot Analysis

Cells are plated at 10⁴ cell cm⁻² and grown under drug treatment for the indicated times. At the end of the treatment period, cells are collected and lysed with 1% SDS in 80 mM Tris-HCL (ph 7.4) buffer supplemented with a proteinase inhibitor cocktail. DNA is fragmented by sonication and protein concentrations are measured by microBCA protein assay kit (Pierce, USA). Samples are mixed with 2x Laemmli sample buffer (Bio-Rad, USA) and run on a SDS-polyacrylamide gel. Gels are transferred to nitrocellulose membranes (Amersham, USA) and probed with anti-KDEL (Stressgen, Canada) (for Grp78 and Grp94); polyclonal anti-CHOP/GADD154 (Santa Cruz, USA), polyclonal anti-erbB2 (DAKO, USA). Following probing, membranes are washed and incubated with an HRP conjugated secondary antibody. Chemiluminesence is detected by exposure to film. Where indicated, membranes are stripped with Stripping Buffer (Pierce, USA) and reprobed with anti-actin (Sigma, USA) primary antibody. To analyze conconavalin A (ConA) binding, the membranes were incubated with 0.2 micro g/ml HRP-conjugated ConA, and chemiluminesence was detected as described.

Results

2-DC and 2-Fluoro-D-Mannose, but not 2-FDG, Kill SKBR3 Cells Growing Under Normoxic Conditions

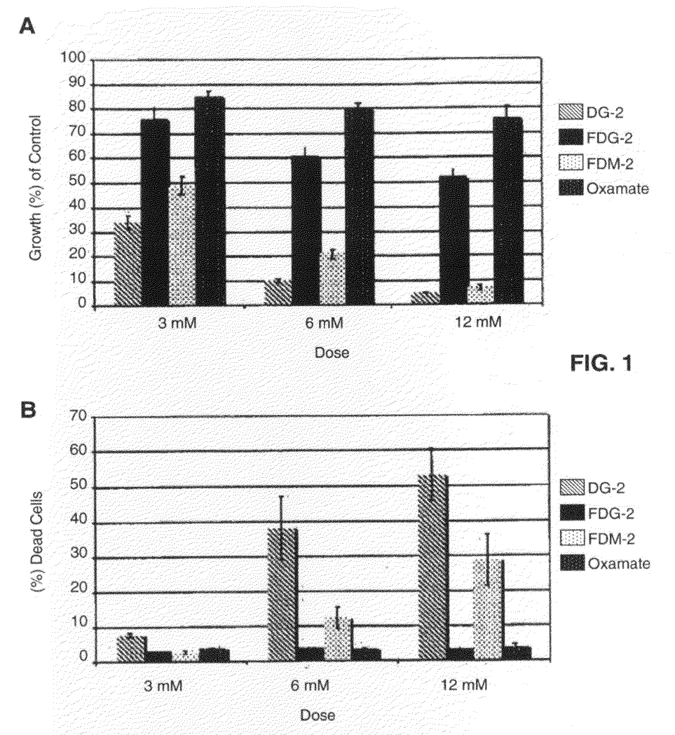

In surveying a number of tumor cell lines for their differential sensitivity to glycolytic inhibitors under normoxic vs hypoxic conditions, it was discovered that the human breast cancer cell line SKBR3 was sensitive to 2-DG when grown under normoxic conditions. FIGS. 1A and B demonstrate that when SKBR3 is treated with 3 mM of 2-DG for 72 hrs, 50% of its growth is inhibited (ID₅₀), while at 12 mM 60% of the cells are killed. Previous studies showed that when mitochondrial respiration is deficient or chemically blocked, tumor cells die when treated with similar doses of 2-DG. Therefore, to determine whether these cells were deficient in mitochondrial respiration, their oxygen consumption was measured. As demonstrated in Table 1 below, there was no significant difference between the average oxygen consumption of SKBR3 cells and two other cell lines that are resistant to 2-DG treatment when grown under normoxic conditions. On the other hand, a mitochondrial deficient cell line, p⁰ showed drastically reduced oxygen consumption, confirming that SKBR3 was respiring normally. Furthermore, two other cell lines, 1420 and HELA, which were sensitive to 2-DG under normoxia, respired as well or better than the resistant cell lines (see Table 1). Thus, the toxicity of 2-DG in these cells under normoxic conditions is due to a mechanism other than blockage of glycolysis. To confirm this, SKBR3 cells were treated with two other glycolytic inhibitors i.e. 2-deoxy-2-fluoro-glucose (2-FDG) and oxamate. In FIGS. 1A and B, it can be seen that neither of these agents caused toxicity to SKBR3 cells when grown under normoxia.

TABLE 1
Comparison of oxygen consumption in 2-DG sensitive vs.
resistant cell lines

| Cell | | Average O₂ consumption |
|---|---|---|
| line | Tissue Type | (nmol/10⁶ cells/min) |
| 143B | Osteosarcoma | 2.81 ± 0.11 |
| P⁰ | Osteosarcoma | 0.09 ± 0.004 |
| SKOV3 | ovarian carcinoma | 2.38 ± 0.32 |
| SKBR3 | breast adenocarcinoma | 2.01 + 0.29 |
| 1420 | pancreatic | 4.70 ± 0.03 |
| | adenocarcinoma | |
| HELA | cervical adenocarcinoma | 2.76 ± 0.04 |

Moreover, 2-fluoro-D-mannose (2-FDM) was similar to 2-DG, albeit less efficient, in causing cytotoxicity in SKBR3 cells (see FIG. 1). Both 2-DG and 2-FDM but not 2-FDG resemble the structure of mannose and thereby can interfere with the metabolism of mannose. This data indicates that interference by 2-DG and 2-FDM with the metabolism of mannose, which is primarily involved in N-linked glycosylation of numerous proteins, results in cell death as well as growth inhibition in SKBR3 cells.

2-FDG is a Better Inhibitor of Glycolysis than 2-DG Leading to Better Depletion of ATP in SKBR3 Cells

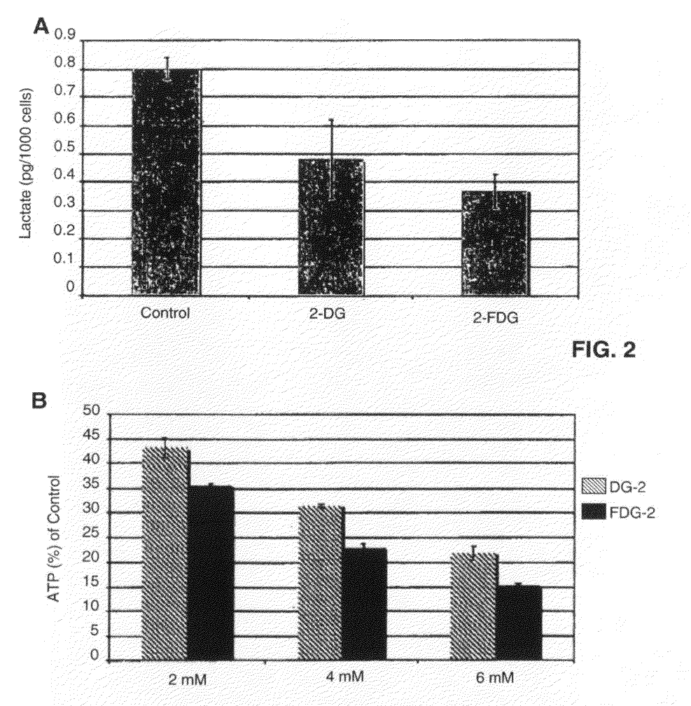

In a previous report, it was suggested that the toxicity of 2-DG in SKBR3 cells growing under normoxia was mediated via inhibition of glycolysis and ATP production. Aft, R. L., et al., Evaluation Of 2-Deoxy-D-Glucose As A Chemotherapeutic Agent: Mechanism Of Cell Death, Br J Cancer 2002;87:805-812. However, as mentioned above, another glycolytic inhibitor, 2-FDG, is non-toxic in these cells. Moreover, the 2-FDG analog is better than 2-DG in inhibiting glycolysis and killing hypoxic cells. Lampidis, T. J., et al., Efficacy of 2-Halogen Substituted D-Glucose Analogs in Blocking Glycolysis and Killing “Hypoxic Tumor Cells,” Cancer Chemother Pharmacol (in press). Indeed, when SKBR3 cells were treated with 2-FDG vs. 2-DG, the former inhibited lactate levels (a measure of glycolysis) better than the latter (see FIG. 2A). Furthermore, ATP depletion was more prominent with 2-FDG treatment, further confirming that this sugar analog is a better inhibitor of glycolysis and ATP production in these cells (see FIG. 2B). Moreover, it was discovered that, when SKBR3 cells were grown under hypoxic conditions, 2-FDG was more toxic than 2-DG, further confirming that it is a better inhibitor of glycolysis in SKBR3 cells (data not shown). Thus, in contrast to previous reports, the toxicity induced by 2-DG under normoxic conditions appears to be independent from its ability to inhibit glycolysis and decrease ATP pools.

2-DG Toxicity in SKBR3 Cells Under Normoxia can be Reversed by Exogenous Mannose

In viral proteins, 2-DO has been shown to inhibit the assembly of N-linked oligosaccharides, and this inhibition can be reversed by exogenous mannose. Datema, R., et al., Interference With Glycosylation Of Glycoproteins, Biochem J 1979;184: 113-123. FIGS. 3A and 3B illustrate that with the addition of mannose, but not other sugars, i.e. glucose, fructose and fucose, cell death from 2-DG exposure under normoxia can be reversed, suggesting that cell death is mediated by interference with glycosylation via a similar mechanism. Datema, R., et al., Interference With Glycosylation Of Glycoproteins, Biochem J 1979;184: 113-123. As a negative control, it was found that mannose does not reverse tunicamycin induced toxicity in SKBR3 cells under the same conditions. This can be explained by the fact that tunicamycin interferes with glycosylation at a step preceding the addition of mannose to the oligosaccharide chain, thereby rendering it independent of mannose metabolism (data not shown).

2-DG Toxicity in Three Models of ‘Hypoxia’ Cannot be Reversed by Exogenous Mannose

As mentioned above, cells growing under hypoxic conditions depend solely on glycolysis to produce energy. Thus, inhibition of this metabolic pathway by glycolytic inhibitors should lead to cell death, as has been previously demonstrated. Maher, J. C., et al., Greater Cell Cycle Inhibition And Cytotoxicity Induced By 2-Deoxy-D-Glucose In Tumor Cells Treated Under Hypoxic vs Aerobic Conditions, Cancer Chemother Pharmacol 2004; 53:116-122. To distinguish the mechanism by which 2-DG is toxic to SKBR3 cells growing under normoxia, mannose was added to cells growing under three different conditions of ‘hypoxia’. As shown in FIGS. 3C and D, no significant difference was found in growth inhibition and cell death in either normal growth medium or in the same medium supplemented with 2 mM mannose. These results provide evidence that the reversal of toxicity of 2-DG in SKBR3 cells growing under normoxia by exogenous mannose is unrelated to the glycolysis, further implicating interference with glycosylation as the mode of cell death in these cells growing under normoxia.

2-DG and 2-FDM are Toxic to Only a Select Number of Tumor Cell Lines Growing Under Normoxic Conditions

To investigate whether the toxicity of 2-DG under normoxic conditions was confined to a certain type of cancer tissue, a number of cell lines were tested. The results of this testing, shown in Table 2, show that only a select number of tumor cell lines (6 out of 15) growing under normal oxygen tension undergo significant cell death when treated with either 2-DG or 2-FDM but not 2-FDG at 6 mM. The cell lines that were found to be sensitive to 2-DG were SKBR3, a breast cancer cell line; 1420, a pancreatic cancer cell line; 2 non-small cell lung cancer cell lines derived directly from patients; RT 8226, a multiple myeloma cell line; HELA, a cervical carcinoma and TG98, a glioblastoma cell line. However, cancer cell lines derived from similar tissues were found to be resistant to both 2-DG and 2-FDM under normal oxygen tension, indicating that toxicity of these sugar analogs is not necessarily tissue type specific.

TABLE 2
Resistant vs. sensitive Cell lines (2-DG under normoxia)

| 2-DG Sensitive Cell Lines | 2-DG Resistant Cell Lines |
|---|---|
| SKBR3, breast cancer | SKOV3, ovarian cancer |
| 1420, pancreatic cancer | 1469, pancreatic cancer |
| HELA, cervical cancer | 143B, osteosarcoma |
| S-1 & S-2, non-small cell | Ra-1,2 and 3, small cell lung cancer |
| lung cancer | |
| TG98, brain cancer (glioblastoma) | MCF-7, breast cancer |
| RT 8228, multiple myeloma | U266, multiple myeloma |
| | HEPA-1, rat hepatoma |
| | MDA-MB-231, breast cancer |
| | MDA-MB-468, breast cancer |

### 2-DG and 2-FDM Decrease Conconavalin A (ConA) Binding and the Molecular Weight of a Glycoprotein in SKBR3 Cells

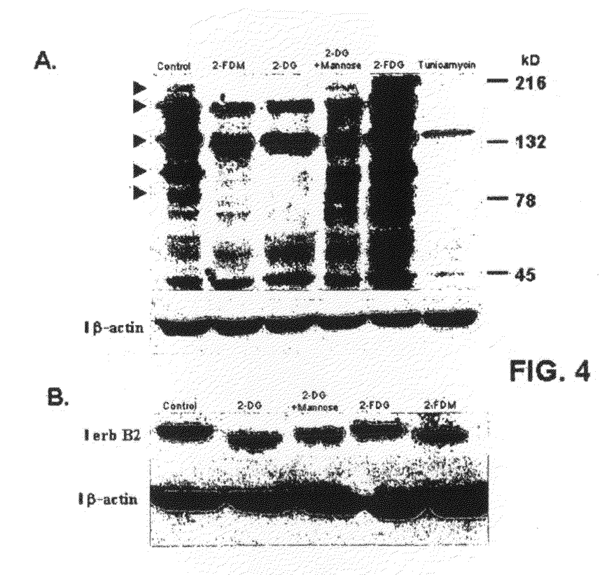

ConA is a lectin that specifically binds mannose on glycoproteins and has been used to detect high mannose type glycoproteins. Protein Purification Methods: A Practical Approach, In: Harris ELV, Angal S, editors. New York: IRLPress at Oxford University Press; 1994. p. 270. This technique was used to show that both 2-DG and 2-FDM as well as tunicamycin decrease ConA binding in a number of glycoproteins (see FIG. 4A). Moreover, exogenous mannose restores control ConA binding levels in 2-DG and 2-FDM but not tunicamycin treated cells, while 2-FDG treated cells show no reduction in ConA binding. Furthermore, change in the size of a known glycoprotein, erbB2, which is a tyrosine-kinase receptor expressed in SKBR3 cells following 2-DG treatment, was analyzed by western blot. FIG. 4B illustrates that both 2-DG and 2-FDM decreased the molecular weight of erbB2, while 2-FDG had no effect. In correlation with the ConA data, exogenous mannose restored the size of the protein to its original weight. These data further support the conclusion that 2-DG and 2-FDM but not 2-FDG are toxic to select tumor cells via interference with N-linked glycosylation, and that this interference can be reversed by mannose.

Treatment by Either 2-DG or 2-FDM Leads to Unfolded Protein Response in SKBR3 Cells Under Normoxia

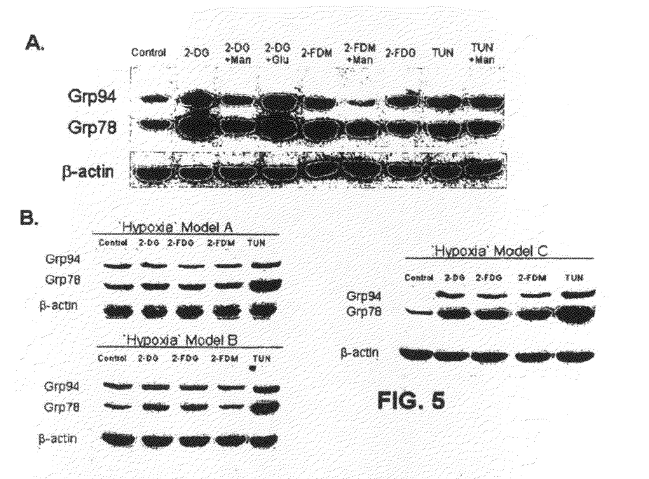

When the normal process of protein glycosylation is affected, misfolded proteins accumulate in the endoplasmic reticulum (ER) leading to a signaling cascade known as unfolded protein response (UPR). Drugs that interfere with glycosylation have been shown to induce UPR, leading to increases in the protein folding capacity of ER via upregulation of chaperones i.e. Grp78TBip or Grp94. As shown in FIG. 5A, when SKBR3 cells are treated with 2-DG, 2-FDM, or tunicamycin, a well-known inhibitor of glycosylation, under normoxia, both Grp78 and Grp94 are upregulated. Moreover, addition of 2 mM mannose reverses the 2-DG and 2-FDM upregulation of chaperones but not that of tunicamycin. The mannose reversal of 2-DG induced UPR correlates with data in FIG. 3D demonstrating that the toxicity of 2-DG is reversed by the addition of exogenous mannose; similar results were found in 2-FDM treated cells (data not shown). As expected, 2-FDG does not increase the levels of these chaperones as much as 2-DG or 2-FDM, correlating with the toxicity data (FIG. 1B) illustrating no cell death in SKBR3 cells when treated under normoxic conditions. In contrast, when 2-DG or 2-FDM are applied to cells growing under three different experimental conditions of hypoxia, no significant upregulation of the UPR is observed in models A and B as compared to model C where both chaperones are upregulated. Moreover, tunicamycin, as a positive control, is shown to induce the synthesis of these chaperones in all three models (FIG. 5B). These results indicate that, when cells are treated with 2-DG or 2-FDM, the mechanism of cell death differs under “hypoxic” (blockage of glycolysis) vs. normoxic (interference with glycosylation) conditions.

Toxicity of 2-DG and 2-FDM Correlates with Induction of the UPR-Specific Apoptotic Pathway in SKBR3 Cells

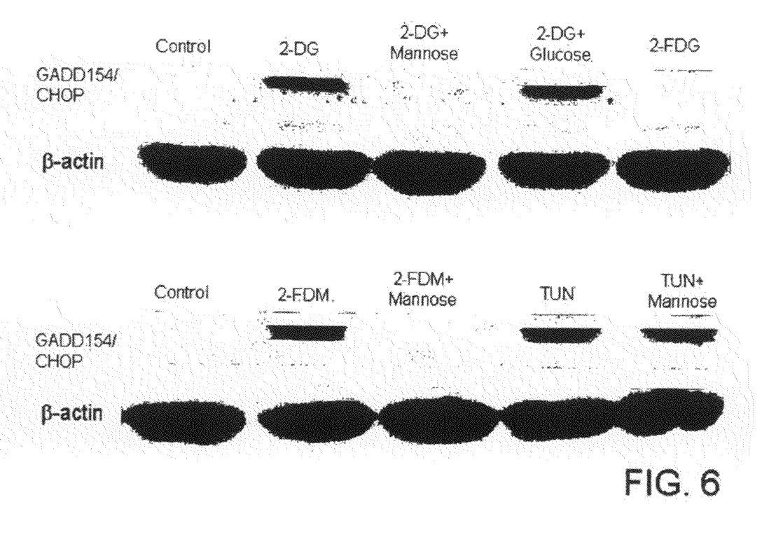

It has been reported that when cells cannot overcome ER stress, UPR induces specific apoptotic pathways via induction of GADD154/CHOP. Xu, C., et al., Endoplasmic Reticulum Stress: Cell Life And Death Decisions, J Clin Invest 2005; 115: 2656-2664; Obeng, E. A., et al., Caspase-12 And Caspase-4 Are Not Required For Caspase-Dependent Endoplasmic Reticulum Stress-Induced Apoptosis, J Biol Chem 2005; 280: 29578-29587. Thus, to determine whether 2-DG and 2-FDM kill SKBR3 cells due to ER stress under normoxia, this UPR-specific apoptotic protein was assayed using western blot analysis. As can be seen in FIG. 6, following 2-DG, 2-FDM, and tunicamycin, but not 2-FDG treatment, GADD154/CHOP is induced. When this apoptotic pathway is induced by either 2-DO or 2-FDM, it can be reversed by co-treatment with mannose; however, tunicamycin induced GADD 154/CHOP cannot be reversed by addition of this sugar. These data correlate with the reversal of cytotoxicity by mannose, as shown in FIG. 2B.

### Discussion of Examples 1 and 2

Solid tumors contain hypoxic as well as normoxic areas due to insufficient angiogenesis, rapid growth of the tumor and decreased oxygen carrying ability of tumor vessels. Gillies, R. J., et al., MRI Of The Tumor Microenviroment, J Magn Reson Imaging 2002; 16:430-450; Maxwell, P. H., et al., Hypoxia-Inducible Facoro-1 Modulates Gene Expression In Solid Tumors And Influences Both Angiogenesis And Tumor Growth, PNAS 1997; 94:8104-8109; Semenza, G. L., Targeting HIF-1 For Cancer Therapy, Nature Rev 2003; 3:721-732. Because the sole energy production pathway in hypoxic cells is glycolysis, it has been shown that the glycolytic inhibitor 2-DG is selectively toxic to these cells but is non-toxic and only growth inhibits aerobic cells. Maher, J. C., et al., Greater Cell Cycle Inhibition And Cytotoxicity Induced By 2-Deoxy-D-Glucose In Tumor Cells Treated Under Hypoxic vs Aerobic Conditions, Cancer Chemother Pharmacol 2004; 53:116-122; Maschek, G., et al., 2-Deoxy-D-Glucose Increases The Efficacy Of Adriamycin And Paclitaxel In Human Osteosarcoma And Non-Small Cell Lung Cancers In Vivo, Cancer Res 2004; 64:31-4; Liu, H., et al., Hypersensitization Of Tumor Cells To Glycolytic Inhibitors, Biochemistry 2001; 40:5542-5547; Liu, H., et al., Hypoxia Increases Tumor Cell Sensitivity To Glycolytic Inhibitors: A Strategy For Solid Tumor Therapy (Model C,. Biochem Pharmacol 2002; 64:1745-1751. However, a select number of tumor cell lines are killed by 2-DG in the presence of oxygen. Among these sensitive cell types is the human breast cancer cell line SKBR3. A deficiency in mitochondrial respiration could explain the sensitivity of these cells to 2-DG, because blockage of glycolysis in cells with compromised mitochondria would lower ATP levels, leading to necrotic cell death. Gramaglia, D., et al., Apoptosis To Necrosis Switching Downstream Of Apoptosome Formation Requires Inhibition Of Both Glycolysis And Oxidative Phosphorylation In A BCL-X_{L} And PKB/AKT-Independent Fashion, Cell Death Differentiation 2004; 11: 342-353. However, this possibility was ruled out by oxygen consumption experiments, which showed that SKBR3 cells respire similarly to two other cells lines found to be resistant to 2-DG under normoxia (Table 1). Furthermore, the rate of respiration of cell line 1420, which is also sensitive to 2-DG under normoxia, was found to be higher than in 2-DO resistant cell lines. Thus, the toxicity of 2-DG in SKBR3 under normoxia cannot be explained by a deficiency in mitochondrial function, indicating that the mechanism of cell death is unrelated to the effect of this sugar on blocking glycolysis.

Previously, it was reported that SKBR3 cells were sensitive to 2-DG under normoxia due to inhibition of glycolysis, leading to depletion of ATP pools which resulted in increased expression of glucose transporter-I and greater uptake of 2-DG. Aft, R. L., et al., Evaluation Of 2-Deoxy-D-Glucose As A Chemotherapeutic Agent: Mechanism Of Cell Death, Br J Cancer 2002; 87:805-812. However, 2-FDG is a more potent inhibitor of glycolysis than 2-DG (11, FIG. 2) but is non-toxic to SKBR3 cells growing under normoxia, further supporting the conclusion that 2-DG kills these cells via a mechanism other than by blockage of glycolysis and inhibition of ATP production.

The data showing that SKBR3 cells are also sensitive to the mannose analog 2-FDM indicates that the manno-configuration of sugar analogs is important for their toxic activity in select tumor cells growing under normoxia. The lack of an oxygen atom at the second carbon of 2-DG renders this compound both a glucose and mannose analog, whereas the fluoro group in 2-FDG renders it a glucose analog only. The conclusion that the manno-configuration is relevant to the toxicity of these sugar analogs is supported by work published in the late 1970s by a group headed by Schwartz.

This group showed that 2-DG, 2-FDG and 2-FDM could interfere with N-linked glycosylation in chick embryo fibroblasts, which were infected with fowl plague virus, resulting in decreased glycoprotein synthesis and viral reproduction. Datema, R., et al., Interference With Glycosylation Of Glycoproteins, Biochem J 1979; 184: 113-123; Datema, R., et al., Formation Of 2-Deoxyglucose-Containing Lipid-Linked Oligosaccharides, Eur J Biochem 1978; 90: 505-516; Datema, R., et al., Fluoro-Glucose Inhibition Of Protein Glycosylation In Vivo, Eur J Biochem 1980; 109:331-341; Schmidt, M. F. G., et al., Nucleoside-diphosphate Derivatives Of 2-Deoxy-D-Glucose In Animal Cells, Eur J Biochem 1974; 49: 237-247; Schmidt, M. F. G., et al., Metabolism Of 2-Deoxy-2-Fluoro-D-[³H] Glucose And 2-Deoxy-2-Fluoro-D-[³H] Mannose In Yeast And Chick-Embryo Cells, Eur J Biochem 1978; 87: 55-68; McDowell, W., et al., Mechanism Of Inhibition Of Protein Glycosylation By The Antiviral Sugar Analogue 2-Deoxy-2-Fluoro-D-Mannose: Inhibition Of Synthesis Of Man(Gicnac)₂ PP-Dol By The Guanosine Diphosphate Ester, Biochemistry 1985; 24:8145-8152. Their reports concluded that 2-DG can inhibit the assembly of lipid linked oligosaccharides, which were to be transferred onto the proteins within the endoplasmic reticulum of the cell. It was demonstrated that a metabolite of 2-DG, GDP-2DG, could cause premature termination of the oligosaccharide assembly leading to shortened lipid-linked oligosaccharides not suitable for their transfer onto proteins. Datema, R., et al., Formation Of 2-Deoxyglucose-Containing Lipid-Linked Oligosaccharides, Eur J Biochem 1978; 90: 505-516. Overall, these results showed that the potency of these analogs to inhibit viral glycoprotein synthesis was in the order of 2-DG>2-FDM>2-FDG, which is similar to the toxicity of these analogs in SKBR3 cells growing under normoxia. Datema, R., et al., Fluoro-Glucose Inhibition Of Protein Glycosylation In Vivo, Eur J Biochem 1980; 109:331-341. This group also reported that the inhibitory effects of these analogs could be reversed by addition of low dose exogenous mannose. Datema, R., et al., Interference With Glycosylation Of Glycoproteins, Biochem J 1979; 184: 113-123. Similarly, 2 mM mannose completely reverses 2-DG and 2-FDM toxicity in SKBR3 cells, indicating that both mannose analogs kill these cells via interfering with N-linked glycosylation. Datema, R., et al., Interference With Glycosylation Of Glycoproteins, Biochem J 1979; 184: 113-123.; Datema, R., et al., Formation Of 2-Deoxyglucose-Containing Lipid-Linked Oligosaccharides, Eur J Biochem 1978; 90: 505-516; Datema, R., et al., Fluoro-Glucose Inhibition Of Protein Glycosylation In Vivo, Eur J Biochem 1980; 109:331-341; Schmidt, M. F. G., et al., Nucleoside-diphosphate Derivatives Of 2-Deoxy-D-Glucose In Animal Cells, Eur J Biochem 1974; 49: 237-247; Schmidt, M. F. G., et al., Metabolism Of 2-Deoxy-2-Fluoro-D-[³H] Glucose And 2-Deoxy-2-Fluoro-D-[³H] Mannose In Yeast And Chick-Embryo Cells, Eur J Biochem 1978; 87: 55-68; McDowell, W., et al., Mechanism Of Inhibition Of Protein Glycosylation By The Antiviral Sugar Analogue 2-Deoxy-2-Fluoro-D-Mannose: Inhibition Of Synthesis Of Man(Gicnac)₂ PP-Dol By The Guanosine Diphosphate Ester, Biochemistry 1985; 24:8145-8152.

Although mannose is a core sugar in N-linked glycosylated proteins, it also can participate in the glycolytic pathway, because it can be converted to fructose-6-phosphate by phosphomannoisomerase. Thus, it remains possible that mannose could reverse the toxicity of 2-DG in SKBR3 cells by circumventing the glycolytic step which 2-DG inhibits (FIG. 7). However, this possibility seems less likely, because 2 mM mannose did not reverse (see FIGS. 3C and 3D) growth inhibition and cell death induced by 2-DG in “hypoxic” models A and B, whereas in model C, in which cells were actually grown under hypoxia, there was a slight recovery effect. This slight recovery could be explained by (1) 2-DG and 2-FDM interfering with glycosylation even under hypoxic conditions, and/or (2) mannose reversing the inhibition of glycolysis in model C, because these cells under 0.5% hypoxia are still undergoing oxidative phosphorylation, albeit reduced. Overall, the reversal of 2-DG and 2-FDM toxicity by mannose in cells sensitive to these sugar analogs under normoxia but not in cells whose mitochondria are shut down (models A and B) supports that interference with glycosylation, and not inhibition of glycolysis, is responsible for the normoxic hypoxia.

When N-linked glycosylation is inhibited, proteins cannot fold properly and are retained in the E R. Ellgaard, L., et al., Quality Control In The Endoplasmic Reticulum, Nat Rev Mol Cell Biol 2003; 4:181-191; Parodi, A. J., Protein Glycosylation And Its Role In Protein Folding, Annu Rev Biochem 2000; 69: 69-93. Accumulation of unfolded proteins results in distention of the organelle as well as perturbed protein translation. In such an event, cells initiate a complex, but yet conserved, signaling cascade, known as unfolded protein response, (UPR) to reestablish homeostasis in the ER. Three ER transmembrane proteins transduce the unfolded protein signal to the nucleus: inositol requiring enzyme 1 (IRE1); double-stranded RNA activated protein kinase (PERK), and activating transcription factor 6 (ATF6). Schroder, M., et al., ER Stress And Unfolded Protein Response, Mutat Res 2005; 569:29-63. When unfolded proteins accumulate in the ER, a molecular chaperone, glucose regulated protein 78 (Grp78/Bip), dissociates from these three ER transmembrane proteins, thereby activating them. Pahl, H. L., Signal Transduction From The Endoplasmic Reticulum To The Cell Nucleus, Physiol Rev 1999; 79: 683-701. This results in a number of metabolic and molecular alterations, including upregulation of sugar transporters, increases in phospholipid synthesis, amino acid transport, and expression of molecular chaperones Grp78/Bip and Grp94. Ma, Y., et al, The Unfolding Tale Of The Unfolded Protein Response, Cell 2001; 107: 827-830; Doerrler. W. T., et al., Regulation Of Dolichol Pathway In Human Fibroblasts By The Endoplasmic Reticulum Unfolded Protein Response, PNAS 1999; 96:13050-13055; Breckenridge, D. G., et al., Regulation Of Apoptosis By Endoplasmic Reticulum Pathways, Oncogene 2003; 22: 8608-8618.

2-DG and 2-FDM upregulate the expression of both Grp78 and Grp94 in SKBR3 cells growing under normoxic conditions, which can be reversed by addition of exogenous mannose, strongly supporting that these sugar analogs are interfering with N-linked glycosylation, leading to unfolded proteins and thereby initiating UPR. Furthermore, 2-FDG, which is a better inhibitor of glycolysis than either 2-DG or 2-FDM, is not as effective in inducing a UPR response. The magnitude of the UPR response to these analogs appears to reflect the degree of interference with glycosylation, which agrees with reports demonstrating that 2-DG>2-FDM>2-FDG in blocking lipid linked oligosaccharide assembly in viral coat proteins. Datema, R., et al., Fluoro-Glucose Inhibition Of Protein Glycosylation In Vivo, Eur J Biochem 1980; 109:331-341; Schmidt, M. F. G., et al., Nucleoside-diphosphate Derivatives Of 2-Deoxy-D-Glucose In Animal Cells, Eur J Biochem 1974; 49: 237-247; Schmidt, M. F. G., et al., Metabolism Of 2-Deoxy-2-Fluoro-D-[³H] Glucose And 2-Deoxy-2-Fluoro-D-[³H] Mannose In Yeast And Chick-Embryo Cells, Eur J Biochem 1978; 87: 55-68; McDowell, W., et al., Mechanism Of Inhibition Of Protein Glycosylation By The Antiviral Sugar Analogue 2-Deoxy-2-Fluoro-D-Mannose: Inhibition Of Synthesis Of Man(Gicnac)₂ PP-Dol By The Guanosine Diphosphate Ester, Biochemistry 1985; 24:8145-8152. Moreover, this UPR data correlates with the cytotoxicity results, which similarly show that 2-DG>2-FDM>>>2-FDG in growth inhibiting and killing SKBR3 cells under normoxia.

On the other hand, in the “hypoxic” models A and B, Grp78 and Grp94 are not upregulated by 2-DG, indicating that these cells die via inhibition of glycolysis and not through interference with glycosylation. A possible mechanism to explain why UPR is not induced in these models relates to levels of ATP known to be necessary for Grp78/Bip binding unfolded proteins and thereby activating UPR. In contrast to model A and B, UPR is induced in model C (FIG. 5B), where ATP levels are decreased less by 2-DG. Moreover, tunicamycin, which is known not to affect ATP levels significantly, does up-regulate the chaperones in the “hypoxic” models, demonstrating a functional UPR pathway in these cells.

UPR is much like p53, where DNA damage signals cell cycle arrest, activation of DNA repair enzymes, and depending on the outcome of these processes, apoptosis. Thus, if UPR fails to establish homeostasis within the endoplasmic reticulum, ER-stress specific apoptotic pathways are activated. Breckenridge, D. G., et al., Regulation Of Apoptosis By Endoplasmic Reticulum Pathways, Oncogene 2003; 22: 8608-8618. Among the mediators of apoptotic pathways which include caspase 4, caspase 12, and CHOP/GADD154, increased activation of the latter has been shown to be a better indicator of the ER-induced mammalian apoptotic pathway than the others. Obeng, E. A., et al., Caspase-12 And Caspase-4 Are Not Required For Caspase-Dependent Endoplasmic Reticulum Stress-Induced Apoptosis, J Biol Chem 2005; 280: 29578-29587. Thus, FIG. 6, where it is shown that expression of CHOP/GADD154 correlates with 2-DG and 2-FDM cytotoxicity in SKBR3 cells growing under normoxia, supports that these sugar analogs are toxic via interference with glycosylation leading to ER stress. Moreover, the reversal of CHOP/GADD154 induction by addition of mannose but not by glucose further supports that 2-DG and 2-FDM are toxic via this mechanism.

A fundamental question is why do certain tumor cell types die when treated with 2-DG in the presence of O₂, whereas most tumor as well as normal cells do not. An answer to this question comes from genetic studies in which the enzyme phosphomannoseisomerase is shown to be deleted in patients suffering from what is described as Carbohydrate-Deficient Glycoprotein Syndrome Type 1b. Niehues, R., et al., Carbohydrate-Deficient Glycoprotein Syndrome Type 1b., J Clin Invest 1998; 101:1414-1420; Freeze, H. H., Human Disorders in N-glycosylation and Animal Models, Biochim Biophys Acta 2002; 1573:388-93. Deletion of this enzyme results in hypoglycosylation of serum glycoproteins, leading to thrombosis and gastrointestinal disorders characterized by protein-losing enteropathy. When exogenous mannose was added to the diets of these patients, their serum glycoproteins returned to normal, their symptoms disappeared. Freeze, H. H., Sweet Solution: Sugars to the Rescue, J Cell Biol 2002; 158:615-616; Paneerselvam, K., et al., Mannose Corrects Altered N-glycosylation in Carbohydrate-Deficient Glycoprotein Syndrome Fibroblasts, J Clin Invest 1996; 97:1478-1487. This correlates with the instant data showing that exogenous mannose rescues the select tumor cells that are killed when treated with 2-DG in normoxia. It is possible that these types of tumor cells are either down-regulating or defective in phosphomannoseisomerase, or that 2-DG effects this enzyme more in these tumor cells than most others which have shown to be resistant to 2-DG treatment in normoxia. However, as indicated in FIG. 7, there are numerous other steps where 2-DG and 2-FDM may be inhibiting mannose metabolism involved with N-linked glycosylation.

2-DG, 2-CM, and 2-FDM (2-FM) kill certain tumor types via interference with glycosylation leading to ER stress and apoptosis. The fmding that 2-FDG does not kill these cells eliminates the possibility that 2-DG and 2-FDM toxicity is due to the inhibition of glycolysis and ATP depletion. These agents can be used as single agent therapies in the treatment of select solid tumors (see FIG. 7).

### Example 3

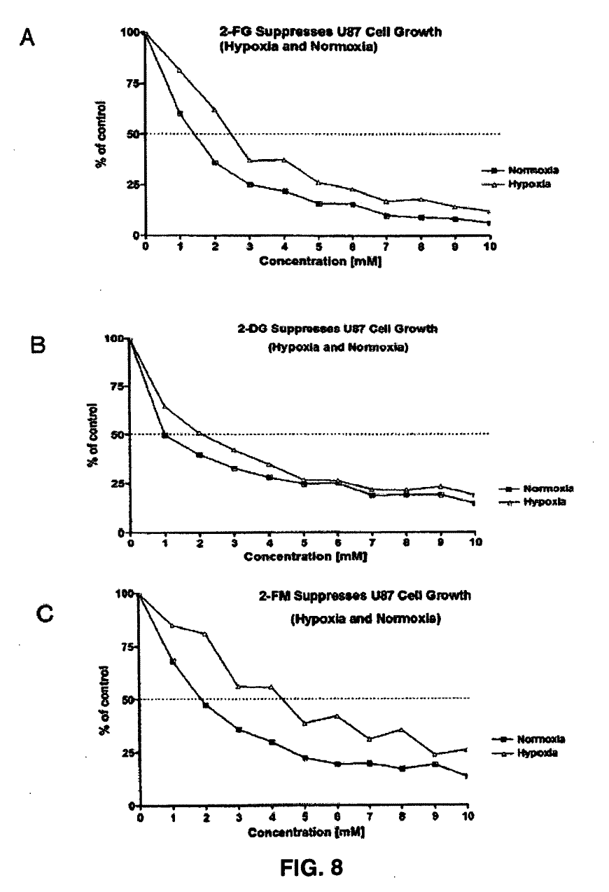

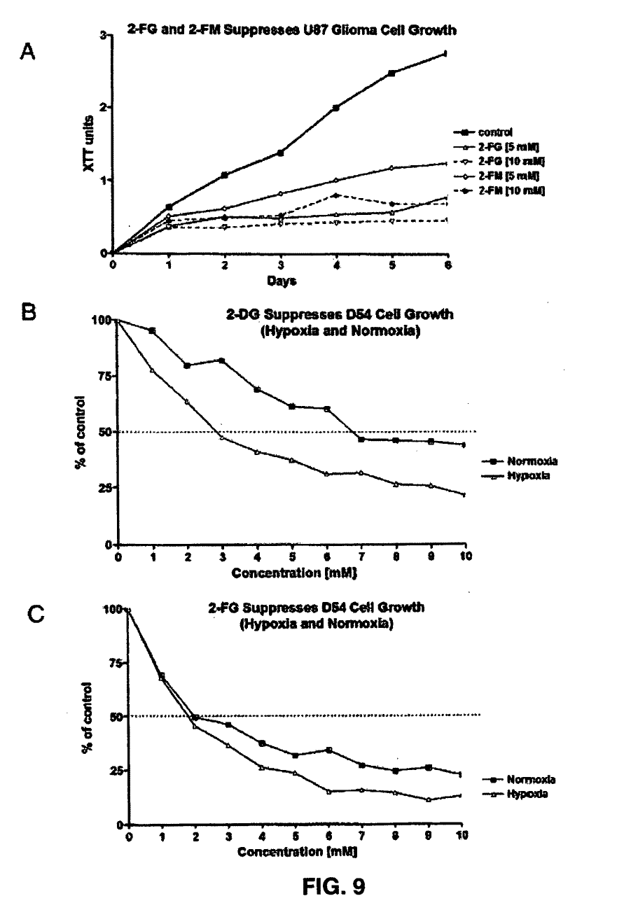

As shown in FIGS. 8 and 9 multiple MTT assays demonstrate the sensitivities of selected high-grade glioma cell lines and various sugar-based glycolytic inhibitors and graphically displayed. Various conditions are used including the exposure to either normoxia or hypoxia and its influence on the sensitivity to these compounds. The results indicate a relatively uniform sensitivity of the various sugar-based glycolytic inhibitors (with some subtle differences). There is a clear difference with some cell lines with respect to the influence of sensitivity in hypoxic conditions. Generally, most cell lines are more sensitive to glycolytic inhibitors when grown in hypoxic conditions, which would be predicted. However, some cell lines such as U87 MG is completely committed to an aerobic glycolysis phenotype (complete “Warburg effect”) that the level of lactic acid (a surrogate marker of glycolysis) is maximal in normoxic conditions and does not increase in hypoxic conditions (see lactate data below). In this circumstance, the difference in sensitivity is explained by the empiric observation that the cells grown in hypoxic conditions are slower growing and therefore probably have less energy demands on the cells.

FIG. 8A shows MIT assays of the U87 human brain tumor cell line being treated with 2-FG in the presence of hypoxia (<1% oxygen) or normoxia (20% oxygen). Both FIGS. 8B and 8C represent similar experiments, however, the sugar-based glycolytic inhibitor is different. In the case of panel B, 2-DG is used and in panel C 2-FM is employed. As can be seen, U87 represents an unusual phenotype that is persistently utilizing glycolysis for its metabolic needs and, therefore, this cell line does not show increased sensitivity to these agents in hypoxia.

FIG. 9 shows growth curves over 6 days in the presence of either 2-FG or 2-FM. This panel demonstrates significant growth inhibition of U87 cell line where 2-FG appears to be slightly more effective than 2-FM. Panel B and panel C demonstrates similar inhibition of growth curves for a cell line D-54 grown both in hypoxia and normoxia conditions. In this case, there is clearly an augmented effect when the cells are grown in hypoxic conditions and this relates to the ability to stimulate further glycolytic metabolism for this particular cell line in hypoxia.

### Example 4

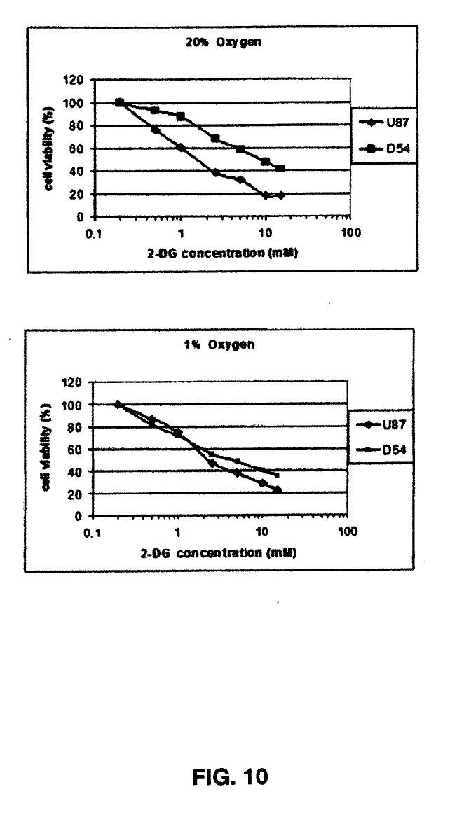

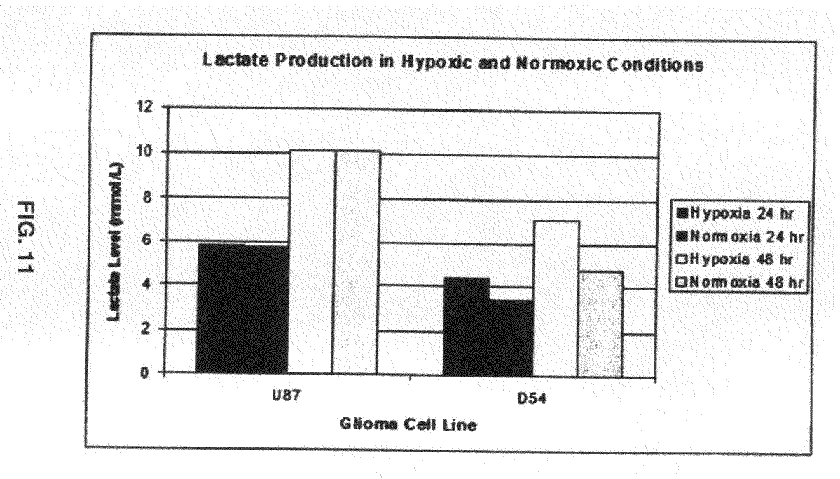

FIGS. 10 and 11 show the difference in sensitivity of the human U87 MG glioblastoma-astrocytoma cell line (U87) versus the D-54 human glioma cell line in normoxia and hypoxic conditions with exposure to 2-DG. U87 MG cells exhibit high rates of glycolysis either in hypoxic conditions or in aerobic conditions (oxidative glycolysis or “The Warburg Effect”), therefore the sensitivity of U87 MG cells to 2-DG does not change when they are grown under hypoxic conditions. On the other hand, D54 cells are partially shifted to glycolytic metabolism under aerobic growth conditions, therefore the sensitivity to 2-DG is greater when this cell line is grown in hypoxic conditions.

FIG. 10 shows the significant difference between these two cells lines and the relative insensitivity of U87, which is more prominent.

FIG. 11 shows the rationale behind this phenotypic difference between U87 and D54. This panel demonstrates the induction of greater glycolysis by D54, whereas U87 is already maximally producing lactate levels.

The results shown in FIGS. 10 and 11 demonstrate a differential effect of hypoxia when the cell lines are treated with glycolytic inhibitors. Cell lines that are highly glycolytically dependent (such as U87 MG) are already maximally sensitized to glycolytic inhibitors and do not require to be in an anoxic environment to show sensitivity. This is demonstrated by the high and unchanging level of lactate production by cell lines such as U87 MG whereas D54 increases both it's sensitivity and lactate levels in response to hypoxia.

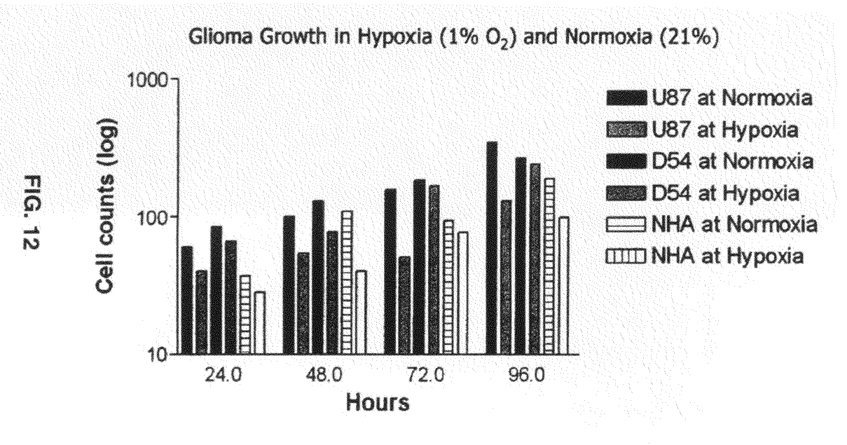

Strikingly, the glioma cell lines are quite resistant to hypoxic conditions. As seen in FIG. 12, cell lines grown in either normoxic conditions or complete hypoxic conditions (<1%) can continue to grow reasonably well relying on glycolysis to provide the energy demands of the cell.

### Example 5

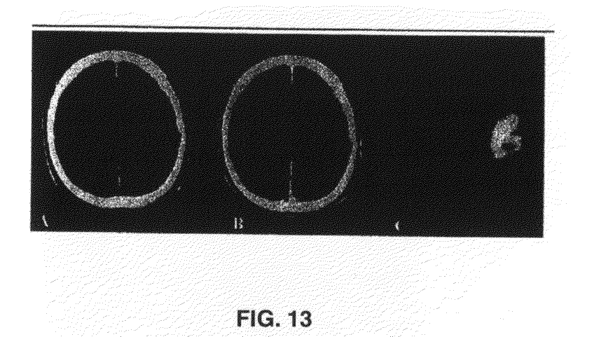

Demonstration of tumor uptake of the 2-DG analog 2-fluoro¹⁸-glucose (2-F¹⁸G). FIG. 13 demonstrates the exaggerated uptake of 2-F¹⁸G within a glioma during routine PET scan studies. A PET scan of a patient with glioblastoma multiforme demonstrates the significant uptake of 2-FG within this tumor. The panels show a CT non-contrast (A), CT with contrast (B) and CT registered PET scan after giving the patient 17 mCi 2-F¹⁸G. This pharmacodynamic phenomena provides a dramatic demonstration that these tumors are uniquely suited for sugar-based glycolysis inhibitors.

### Example 6

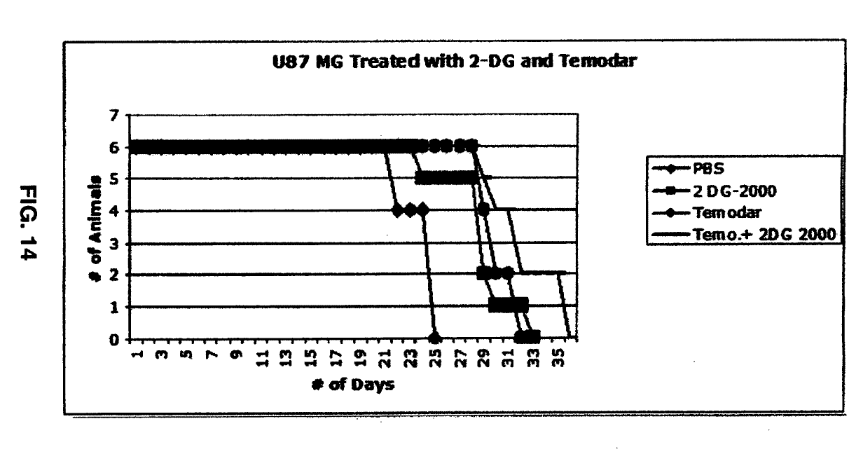

Treatment of human gliomas in mice. Mouse orthotopic xenografts of human glioma cells were treated with either 2-DG alone or with Temozolomide (Temodar). These animals represent an orthotopic xenograft model of high-grade glioma. These experiments were repeated three times with similar results as show in FIG. 14. Animals were implanted intracranially with U87 MG cells and were then treated after 5 days with either negative control (PBS), positive control (Temodar), experimental single agent (2-DG) or experimental combination (2-DG+Temodar).

The results shown in FIG. 12 demonstrate for the first time single agent efficacy of 2-DG against an orthotopic tumor model. These results were repeated and consistent in three consecutive animal experiments with a total of 18 animals in each group (data not shown). This particular animal model is very stringent and only modest gains in survival are ever revealed with investigational new drugs. As can be seen 2-DG is equally effect as the best drug currently available for brain tumors, Temozolomide (seen as positive control).

Surprisingly, 2-DG was equally effective as Temodar and the combination was even superior to single agent therapy. 2-DG was given orally and was well tolerated. The functional equivalence of 2-DG and Temodar was notable because Temodar is the current “gold standard” for treatment of brain tumors. Finally, single agent efficacy has also been demonstrated for this class of agents, which is unique for this disease.

These results demonstrated that an inhibition of glycolysis increased animal survival similarly to that of the positive control used, temozolomide. This therapy was well tolerated by the animals and showed no evidence of toxicity. Finally, we are now selecting compounds from a group of related sugar-based glycolytic inhibitors for lead candidate selections, which will be based on in vitro and in vivo efficacy, pharmacokinetic properties, chemical stability and the cost of chemical synthesis. Upon lead selection, more advanced studies as well as formal animal toxicology testing will be initiated.

While specific embodiments of the invention have been shown and described in detail to illustrate the application of the principles of the invention, it will be understood that the invention may be embodied otherwise without departing from such principles.

1. A method of treating glioblastoma comprising the step of administering a therapeutically effective amount of 2-FM, 2-CM or 2-BM to a subject in need thereof.

2. A method of treating pancreatic cancer comprising the step of administering a therapeutically effective amount of 2-FM, 2-CM or 2-BM to a subject in need thereof.

3. A method of treating the proliferation of tumors comprising the step of administering a therapeutically effective amount of 2-FM, 2-CM or 2-BM to a subject in need thereof.

4. A method of treatment of cancer comprising the step of administering a therapeutically effective amount of 2-FM, 2-CM or 2-BM to a subject in need thereof, wherein cancer cell death occurs by autophagy.

5. (canceled)

6. A method for achieving an effect in a patient comprising the administration of a therapeutically effective amount 2-FM, 2-CM or 2-BM wherein the effect is selected from cell death of pancreatic cancer cells and cell death of glioblastoma cells.

7. A method of treating high grade, highly glycolic gliomas comprising the step of administering a therapeutically effective amount of 2-DG to the gliomas wherein cell death of the gliomas occurs.
